(19) United States
(12) Patent Application Publication
(10) Pub. No.: US 2002/0082270 A1
(43) Pub. Date: Jun. 27, 2002

(54) Aminoquinazolines which inhibit signal transduction mediated by tyrosine kinases

(76) Inventors: Frank Himmelsbach, Mittelbiberach (DE); Elke Langkopf, Warthausen (DE); Birgit Jung, Schwabenheim (DE); Stefan Blech, Warthausen (DE); Flavio Solca, Wien (AT)

Correspondence Address: BOEHRINGER INGELHEIM CORPORATION, 900 RIDGEBURY ROAD, P. O. BOX 368, RIDGEFIELD, CT, 06877, US

(21) Appl. No.: 09/934,753

(22) Filed: Aug. 22, 2001

Related U.S. Application Data
(60) Provisional application No. 60/230,119, filed on Sep. 5, 2000.

(30) Foreign Application Priority Data
Aug. 26, 2000 (DE) DE 100 42 061.3

(51) Int. Cl. A61K 31/517; A61K 31/4709; A61K 31/47; C 07D 4 3/02; C 07D 4 1/02; C07D 239/72; C07D 215/38

(52) U.S. Cl. 514/266.2; 514/266.23; 514/266.4; 514/313; 544/284; 544/293; 546/159

(57) ABSTRACT

Compounds of the formula

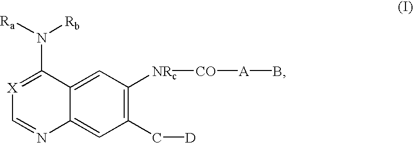

having an inhibitory effect on signal transduction mediated by tyrosinekinases, the use thereof for treating diseases, particularly tumoral diseases, diseases of the lungs and respiratory tract, and the preparation thereof.

### RELATED APPLICATIONS

Benefit of U.S. Provisional Application Ser. No. 60/230,119, filed on Sep. 5, 2000 is hereby claimed.

### DESCRIPTION OF THE INVENTION

The present invention relates to bicyclic heterocycles of general formula

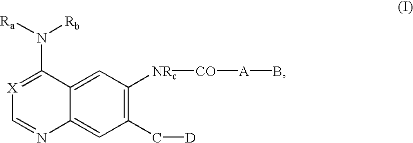

the tautomers, the stereoisomers and the salts thereof, particularly the physiologically acceptable salts thereof with inorganic or organic acids or bases which have valuable pharmacological properties, particularly an inhibitory effect on signal transduction mediated by tyrosine kinases, the use thereof for treating diseases, particularly tumoral diseases, diseases of the lungs and respiratory tract, and the preparation thereof.

In the above general formula I

Rₐ denotes a hydrogen atom or a methyl group,

R_{b} denotes a phenyl, benzyl or 1-phenylethyl group, wherein the phenyl core is substituted in each case by the groups R₁ to R₃, whilst

R₁ and R₂, which may be identical or different, each denote a hydrogen, fluorine, chlorine, bromine or iodine atom,

a methyl, ethyl, hydroxy, methoxy, ethoxy, amino, cyano, vinyl or ethynyl group,

an aryl, aryloxy, arylmethyl or arylmethoxy group,

a methyl or methoxy group substituted by 1 to 3 fluorine atoms or _{R}₁ together with R₂, if they are bound to adjacent carbon atoms, denote a —CH═CH—CH═CH—, —CH═CH—NH— or —CH═N—NH— group and

R₃ denotes a hydrogen, fluorine, chlorine or bromine atom,

R_{c} denotes a hydrogen atom or a methyl group,

X denotes a methyne group substituted by a cyano group or a nitrogen atom,

A denotes a 1,1- or 1,2-vinylene group, each of which may be substituted by one or two methyl groups or by a trifluoromethyl group,

an ethynylene group, or

a 1,3-butadien-1,4-ylene group optionally substituted by a methyl or trifluoromethyl group,

B denotes a hydrogen atom or a C₁₋₄-alkyl group, a methyl group substituted by I to 3 fluorine atoms, an ethyl group substituted by 1 to 5 fluorine atoms, a C₁₋₄-alkylcarbonyl, carboxy, C₁₋₄-alkoxycarbonyl, aminocarbonyl, C₁₋₄-alkylaminocarbonyl, di-(C₁₋₄-alkyl)-aminocarbonyl, pyrrolidinocarbonyl, piperidinocarbonyl, morpholinocarbonyl or a 4-(C₁₋₄-alkyl)-piperazinocarbonyl group, or

a C₁₋₄-alkyl group substituted by the group R₄, whilst R₄ denotes a C₁₋₄-alkoxy group,

an amino group substituted by two C₁₋₄-alkyl groups, wherein the alkyl groups may be identical or different and each alkyl moiety may be substituted from position 2 by a C₁₋₄-alkoxy- or di-(C₁₋₄-alkyl)-amino group or by a 4- to 7-membered alkyleneimino group, whilst in the above-mentioned 6- to 7-membered alkyleneimino groups in each case a methylene group may be replaced in the 4-position by an oxygen or sulphur atom, by a sulphinyl, sulphonyl or N-(C₁₋₄-alkyl)-imino group,

a 4- to 7-membered alkyleneimino group optionally substituted by 1 to 4 methyl groups,

a 6- to 7-membered alkyleneimino group optionally substituted by 1 or 2 methyl groups, wherein in each case a methylene group in the 4-position is replaced by an oxygen or sulphur atom, by a sulphinyl, sulphonyl or N-(C₁₋₂-alkyl)-imino group, or

an imidazolyl group optionally substituted by 1 to 3 methyl groups,

C denotes a C₁₋₆-alkylene group, a —O—C₁₋₆-alkylene group, whilst the alkylene moiety is linked to the group D, or an oxygen atom, which may not be linked to a nitrogen atom of the group D, and

D denotes a pyrrolidino group in which the two hydrogen atoms are replaced in the 2-position by a group E, wherein

E denotes a —CH₂—O—CO—CH₂—, —CH₂CH₂—O—CO—, —CH₂—O—CO—CH₂CH₂—, —CH₂CH₂—O—CO—CH₂— or —CH₂CH₂CH₂—O—CO— bridge optionally substituted by one or two C₁₋₂-alkyl groups,

a pyrrolidino group in which the two hydrogen atoms are replaced in the 3-position by a group F, wherein

F denotes a —O—CO—CH₂CH₂—, —CH₂—O—CO—CH₂—, —CH₂CH₂—O—CO—, —O—CO—CH₂CH₂CH₂—, —CH₂—O—CO—CH₂CH₂—, —CH₂CH₂—O—CO—CH₂—, —CH₂CH₂CH₂—O—CO—, —O—CO—CH₂—NR₅—CH₂—, —CH₂—O—CO—CH₂—NR₅—, —O—CO—CH₂—O—CH₂— or —CH₂—O—CO—CH₂—O—bridge optionally substituted by one or two C₁₋₂-alkyl groups, whilst

R₅ denotes a hydrogen atom or a C₁₋₄-alkyl group,

a piperidino or hexahydroazepino group, wherein the two hydrogen atoms are replaced in the 2-position by a group E, where E is as hereinbefore defined,

a piperidino or hexahydroazepino group, wherein in each case the two hydrogen atoms in the 3-position or in the 4-position are replaced by a group F, where F is as hereinbefore defined,

a piperazino- or 4-(C₁₋₄-alkyl)-piperazino group, wherein the two hydrogen atoms in the 2-position or in the 3-position of the piperazino ring are replaced by a group E, where E is as hereinbefore defined,

a pyrrolidino or piperidino group, wherein two vicinal hydrogen atoms are replaced by a —O—CO—CH₂—, —CH₂—O—CO—, —O—CO—CH₂CH₂—, —CH₂—O—CO—CH₂—, —CH₂CH₂—O—CO—, —O——CO—CH₂—NR₅— or —O—CO—CH₂—O— bridge optionally substituted by one or two C-₁₋₂-alkyl groups, whilst R₅ is as hereinbefore defined and the heteroatoms of the above-mentioned bridges are not bound to the 2- or 5-position of the pyrrolidino ring and are not bound to the 2- or 6-position of the piperidino ring,

a piperazino or 4-(C₁₋₄-alkyl)-piperazino group, wherein a hydrogen atom in the 2-position together with a hydrogen atom in the 3-position of the piperazino ring are replaced by a —CH₂—O—CO—CH₂— or —CH₂CH₂—O—CO— bridge optionally substituted by one or two C₁₋₂-alkyl groups,

a piperazino group in which a hydrogen atom in the 3-position together with the hydrogen atom in the 4-position are replaced by a —CO—O—CH₂CH₂— or —CH₂—O—CO—CH₂— bridge optionally substituted by one or two C₁₋₂-alkyl groups, whilst in each case the left-hand end of the above-mentioned bridges is bound to the 3-position of the piperazino ring,

a pyrrolidino, piperidino or hexahydroazepino group substituted by the group R₆, wherein

R₆ denotes a 2-oxo-tetrahydrofaranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups,

a pyrrolidino group substituted in the 3-position by a 2-oxo-morpholino group, whilst the 2-oxo-morpholino group may be substituted by one or two C₁₋₂-alkyl groups,

a piperidino or hexahydroazepino group substituted in the 3- or 4-position by a 2-oxo-morpholino group, whilst the 2-oxo-morpholino group may be substituted by one or two C₁₋₂-alkyl groups,

a 4-(C₁₋₄-alkyl)-piperazino or 4-(C₁₋₄-alkyl)-homopiperazino group substituted at a ring nitrogen atom by R₆, wherein R₆ is as hereinbefore defined,

a piperazino or homopiperazino group substituted in the 4-position by the group R₇, wherein

R₇ denotes a 2-oxo-tetrahydrofuran-3-yl, 2-oxo-tetrahydrofuran-4-yl, 2-oxo-tetrahydropyran-3-yl, 2-oxo-tetrahydropyran-4-yl or 2-oxo-tetrahydropyran-5-yl group optionally substituted by one or two C₁₋₂-alkyl groups,

a pyrrolidino group substituted in the 3-position by a (R₅NR₇)—, R₇O—, R₇S—, R₇SO— or R₇SO₂— group, whilst R₅ and R₇ are as hereinbefore defined,

a piperidino or hexahydroazepino group substituted in the 3- or 4-position by a (R₅NR₇)—, R₇O—, R₇S—, R₇SO— or R₇SO₂— group, wherein R₅ and R₇ are as hereinbefore defined,

a pyrrolidino, piperidino or hexahydroazepino group substituted by a R₆-C₁₋₄-alkyl-, (R₅NR₇)-C₁₋₄-alkyl-, R₇O—C₁₋₄-alkyl-, R₇S—C₁₋₄-alkyl-, R₇SO—C₁₋₄-alkyl-, R₇SO₂-C₁₋₄-alkyl- or (R₅NR₇)—CO— group, wherein R₅ to R₇ are as hereinbefore defined,

a pyrrolidino group substituted in the 3-position by a R₆—CO—NR₄, R₆-C₁₋₄-alkylene-CONR₄, (R₅NR₇)—C₁₋₄-alkylene-CONR₅, R₇O—C₁₋₄-alkylene-CONR₅, R₇S—C₁₋₄-alkylene-CONR₅, R₇SO—C₁₋₄-alkylene-CONR₅, R₇SO₂-C₁₋₄-alkylene-CONR₅, 2-oxo-morpholino-C₁₋₄-alkylene-CONR₅, R₆-C₁₋₄-alkylene-Y or C₂₋₄-alkyl-Y group, whilst the C₂₋₄-alkyl moiety of the C₂₋₄-alkyl-Y group is substituted in each case from position 2 by a (R₅NR₇)—, R₇O—, R₇S—, R₇SO— or R₇SO₂— group and the 2-oxo-morpholino moiety may be substituted by one or two C₁₋₂-alkyl groups, wherein

R₅ to R₇ are as hereinbefore defined and Y denotes an oxygen or sulphur atom, an imino, N—(C₁₋₄-alkyl)-imino, sulphinyl or sulphonyl group,

a piperidino- or hexahydroazepino group substituted in the 3- or 4-position by a R₆—CO—NR₅, R₆—C₁₋₄-alkylene-CONR₅, (R₅NR₇)—C₁₋₄-alkylene-CONR₅, R₇O—C₁₋₄-alkylene-CONR₅, R₇S— C₁₋₄-alkylene-CONR₅, R₇SO— C₁₋₄-alkylene-CONR₅, R₇SO₂- C₁₋₄-alkylene-CONR₅, 2-oxo-morpholino-C₁₋₄-alkylene-CONR₅, R₆—C₁₋₄-alkylene-Y or C₂₋₄-alkyl-Y group, wherein Y is as hereinbefore defined, the 2-oxo-morpholino moiety may be substituted by one or two C₁₋₂-alkyl groups and the C₂₋₄-alkyl moiety of the C₂₋₄-alkyl-Y group is substituted in each case from position 2 by a (R₅NR₇)—, R₇O—, R₇S—, R₇SO— or R₇SO₂— group, whilst R₅ to R₇ are as hereinbefore defined,

a 4-(C₁₋₄-alkyl)-piperazino or 4-(C₁₋₄-alkyl)-homopiperazino group substituted at a ring nitrogen atom by a R₆—C₁₋₄-alkyl-, (R₅NR₇)-C₁₋₄-alkyl-, R₇O—C₁₋₄-alkyl-, R₇S—C₁₋₄-alkyl-, R₇SO—C₁₋₄-alkyl-, R₇SO₂—C₁₋₄-alkyl- or R₅NR₇—CO— group, wherein R₅ to R₇ are as hereinbefore defined,

a piperazino or homopiperazino group substituted in the 4-position by a R₆-C₁₋₄-alkyl-, R₆—CO—, R₆—C₁₋₄-alkylene-CO—, (R₅NR₇)-C₁₋₄-alkylene-CO—, R₇O—C₁₋₄-alkylene-CO—, R₇S—C₁₋₄-alkylene-CO—, R₇SO—C₁₋₄-alkylene-CO— or R₇SO₂—C₁₋₄-alkylene-CO— group, wherein R₅ to R₇ are as hereinbefore defined,

a piperazino or homopiperazino group substituted in the 4-position by a C₂₋₄-alkyl group, wherein the C₂₋₄-alkyl group is substituted in each case from position 2 by a (R₅NR₇)—, R₇O—, R₇S—, R₇SO— or R₇SO₂— group, whilst R₅ and R₇ are as hereinbefore defined,

a pyrrolidino, piperidino- or hexahydroazepino group substituted by a 2-oxo-morpholino-C₁₋₄-alkyl group, wherein the 2-oxo-morpholino moiety may be substituted by one or two C₁₋₂-alkyl groups,

a pyrrolidino group substituted in the 3-position by a C₂₋₄-alkyl-Y group, wherein Y is as hereinbefore defined and the C₂₋₄-alkyl moiety of the C₂₋₄-alkyl-Y group is substituted in each case from position 2 by a 2-oxo-morpholino group optionally substituted by one or two C₁₋₂-alkyl groups,

a piperidino or hexahydroazepino group substituted in the 3- or 4-position by a C₂₋₄-alkyl-Y group, wherein Y is as hereinbefore defined and the C₂₋₄-alkyl moiety of the C₂₋₄-alkyl-Y group is substituted in each case from position 2 by a 2-oxo-morpholino group optionally substituted by one or two C₁₋₂-alkyl groups,

a 4-(C₁₋₄-alkyl)-piperazino- or 4-(C₁₋₄-alkyl)-homopiperazino group substituted at a ring nitrogen atom by a 2-oxo-morpholino-C₁₋₄-alkyl group, wherein the 2-oxo-morpholino moiety may be substituted by one or two C₁₋₂-alkyl groups,

a piperazino or homopiperazino group substituted in the 4-position by a 2-oxo-morpholino-C₁₋₄-alkylene-CO group, wherein the 2-oxo-morpholino moiety may be substituted by one or two C₁₋₂-alkyl groups,

a piperazino or homopiperazino group substituted in the 4-position by a C₂₋₄-alkyl group, wherein the C₂₋₄-alkyl moiety is substituted in each case from position 2 by a 2-oxo-morpholino group optionally substituted by one or two C₁₋₂-alkyl groups,

a pyrrolidinyl or piperidinyl group substituted in the 1-position by the group R₇, by a R₆-C₁₋₄-alkyl-, R₆—CO—, R₆-C₁₋₄-alkylene-CO—, (R₅NR₇)-C₁₋₄-alkylene-CO—, R₇O—C₁₋₄-alkylene-CO—, R₇S—C₁₋₄-alkylene-CO—, R₇SO—C₁₋₄-alkylene-CO—, R₇SO₂—C₁₋₄-alkylene-CO— or 2-oxo-morpholino-C₁₋₄-alkylene-CO— group, wherein R₅ to R₇ are as hereinbefore defined and the 2-oxo-morpholino moiety may be substituted by one or two C₁₋₂-alkyl groups,

a pyrrolidinyl or piperidinyl group substituted in the 1-position by a C₂₋₄-alkyl group, wherein the C₂₋₄-alkyl moiety is substituted in each case from position 2 by a (R₅NR₇)—, R₇O—, R₇S—, R₇SO—, R₇SO₂— or 2-oxo-morpholino group, whilst R₅ and R₇ are as hereinbefore defined and the 2-oxo-morpholino moiety may be substituted by one or two C₁₋₂-alkyl groups,

a pyrrolidin-3-yl-NR₅, piperidin-3-yl-NR₅ or piperidin-4-yl-NR₅ group substituted at the ring nitrogen atom in each case by the group R₇, by a R₆—C₁₋₄-alkyl-, R₆—CO—, R₆-C₁₋₄-alkylene-CO—, (R₅NR₇)—C₁₋₄-alkylene-CO—, R₇O—C₁₋₄-alkylene-CO—, R₇S—C₁₋₄-alkylene-CO—, R₇SO—C₁₋₄-alkylene-CO—, R₇SO₂-C₁₋₄-alkylene-CO— or 2-oxo-morpholino-C₁₋₄-alkylene-CO— group, wherein R₅ to R₇ are as hereinbefore defined and the 2-oxo-morpholino moiety may be substituted by one or two C₁₋₂-alkyl groups,

a pyrrolidin-3-yl-NR₅, piperidin-3-yl-NR₅ or piperidin-4-yl-NR₅ group substituted in each case at the ring nitrogen atom by a C₂₋₄-alkyl group, wherein the C₂₋₄-alkyl moiety is substituted in each case from position 2 by a (R₅NR₇)—, R₇O—, R₇S—, R₇SO—, R₇SO₂— or 2-oxo-morpholino group, whilst R₅ and R₇ are as hereinbefore defined and the 2-oxo-morpholino moiety may be substituted by one or two C₁₋₂-alkyl groups,

a R₆-C₁₋₄-alkylene-NR₅ group in which R₅ and R₆ are as hereinbefore defined, or

a C₂₋₄-alkyl-NR₄ group, wherein the C₂₋₄-alkyl moiety is substituted in each case from position 2 by a (R₅NR₇)—, R₇O—, R₇S—, R₇SO—, R₇SO₂— or 2-oxo-morpholino group, whilst R₅ and R₇ are as hereinbefore defined and the 2-oxo-morpholino moiety may be substituted by one or two C₁₋₂-alkyl groups,

a 2-oxo-morpholin-4-yl group substituted by the group R₈ or by the group R₈ and a C₁₋₄-alkyl group, whilst

R₈ denotes a C₃₋₄-alkyl, hydroxy-C₁₋₄-alkyl, C₁₋₄-alkoxy-C₁₋₄-alkyl, di-(C₁₋₄-alkyl)-amino-C₁₋₄-alkyl, pyrrolidino-C₁₋₄-alkyl, piperidino-C₁₋₄-alkyl, morpholino-C₁₋₄-alkyl, 4-(C₁₋₄-alkyl)-piperazino-C₁₋₄-alkyl, C₁₋₄-alkylsulphanyl-C₁₋₄-alkyl, C₁₋₄-alkylsulphinyl-C₁₋₄-alkyl, C₁₋₄-alkylsulphonyl-C₁₋₄-alkyl, cyan-C₁₋₄-alkyl, C₁₋₄-alkoxycarbonyl-C₁₋₄-alkyl, aminocarbonyl-C₁₋₄-alkyl, C₁₋₄-alkyl-aminocarbonyl-C₁₋₄-alkyl, di-(C₁₋₄-alkyl)-aminocarbonyl-C₁₋₄-alkyl, pyrrolidinocarbonyl-C₁₋₄-alkyl, piperidinocarbonyl-C₁₋₄-alkyl, morpholinocarbonyl-C₁₋₄-alkyl or a 4-(C₁₋₄-alkyl)-piperazinocarbonyl-C₁₋₄-alkyl group,

a 2-oxo-morpholin-4-yl group substituted by two groups R₈, whilst R₈ is as hereinbefore defined and the two groups R₈ may be identical or different,

a 2-oxo-morpholin-4-yl group in which the two hydrogen atoms of a methylene group are replaced by a —(CH₂)ₘ—, —CH₂—Y—CH₂—, —CH₂—Y—CH₂—CH₂—, —CH₂CH₂—Y—CH₂CH₂— or —CH₂CH₂—Y—CH₂CH₂CH₂— bridge optionally substituted by one or two C₁₋₂-alkyl groups, whilst

m denotes the number 2, 3, 4, 5 or 6 and

Y denotes an oxygen or sulphur atom, a sulphinyl, sulphonyl or C₁₋₄-alkylimino group,

a 2-oxo-morpholin-4-yl group in which a hydrogen atom in the 5-position together with a hydrogen atom in the 6-position is replaced by a —(CH₂)ₙ—, —CH₂—Y—CH₂—, —CH₂—Y—CH₂CH₂— or —CH₂—CH₂—Y—CH₂— bridge, whilst

Y is as hereinbefore defined and

n denotes the number 2, 3 or 4,

whilst, unless otherwise stated, the aryl moieties mentioned in the definitions of the above-mentioned groups denote a phenyl group which may be mono- or disubstituted by R₉, whilst the substituents may be identical or different and

R₉ denotes a fluorine, chlorine, bromine or iodine atom, a C₁₋₂-alkyl, trifluoromethyl or C₁₋₂-alkoxy group, or

two groups R₉, if they are bound to adjacent carbon atoms, together denote a C₃₋₄-alkylene, methylenedioxy or 1,3-butadien-1,4-ylene group.

Preferred compounds of the above general formula I are those wherein

Rₐ denotes a hydrogen atom,

R_{b} denotes a 1-phenylethyl, 3-methylphenyl, 3-chlorophenyl, 3-bromophenyl or 3-chloro-4-fluorophenyl group,

R_{c} denotes a hydrogen atom,

X denotes a nitrogen atom,

A denotes a 1,2-vinylene or ethynylene group,

B denotes a hydrogen atom,

C denotes an —O—CH₂CH₂—, —O—CH₂CH₂CH₂— or —O—CH₂CH₂CH₂CH₂— group, whilst the alkylene moiety in each case is linked to the group D, and

D denotes a piperidino group in which the two hydrogen atoms in the 4-position are replaced by a —CH₂—O—CO—CH₂—, —CH₂H₂—O—CO—, —CH₂CH₂—O—CO—CH₂—, —O—CO—CH₂—NCH₃—CH₂— or —O—CO—CH₂—O—CH₂—bridge,

a piperazino group in which a hydrogen atom in the 3-position together with the hydrogen atom in the 4-position are replaced by a —CO—O—CH₂—CH₂— or —CH₂—O—CO—CH₂— bridge, whilst in each case the left-hand ends of the above-mentioned bridges are bound to the 3-position of the piperazino ring,

a piperidino group which is substituted in the 4-position by a 2-oxo-morpholino or 2-oxo-morpholinomethyl group, whilst the 2-oxo-morpholino moiety may be substituted in each case by one or two methyl groups,

a piperazino group which is substituted in the 4-position by a 2-oxo-tetrahydrofuran-3-yl- or 2-oxo-tetrahydrofuran-4-yl group,

a piperidino group which is substituted in the 4-position by a R₆S group, whilst

R₆ denotes a 2-oxo-tetrahydrofuran-3-yl or 2-oxo-tetrahydrofuran-4-yl group,

a piperazino group which is substituted in the 4-position by a 2-oxo-tetrahydrofuranylmethyl or 2-oxo-tetrahydrofuranyl-carbonyl group,

a piperazino group which is substituted in the 4-position by a [2-(2-oxo-tetrahydrofuran-3-ylsulphenyl)ethyl] group,

a piperidin-4-yl group which is substituted in the 1-position by a 2-oxo-tetrahydrofiran-3-yl or 2-oxo-tetrahydrofuran-4-yl group,

a 2-oxo-morpholin-4-yl group which is substituted by a methoxymethyl or methoxyethyl group,

a 2-oxo-morpholin-4-yl group in which the two hydrogen atoms of a methylene group are replaced by a —CH₂CH₂CH₂CH₂—, —CH₂CH₂CH₂CH₂CH₂—, —CH₂—O—CH₂CH₂— or —CH₂CH₂—O—CH₂CH₂— bridge,

the tautomers, stereoisomers and the salts thereof.

Particularly preferred compounds of the above general formula I are those wherein

Rₐ denotes a hydrogen atom,

R_{b} denotes a 1-phenylethyl or 3-chloro-4-fluorophenyl group,

R_{c} denotes a hydrogen atom,

X denotes a nitrogen atom,

A denotes a 1,2-vinylene group,

B denotes a hydrogen atom,

C denotes an —O—CH₂CH₂—, —O—CH₂CH₂CH₂— or —O—CH₂CH₂CH₂CH₂— group, whilst the alkylene moiety in each case is linked to the group D, and

D denotes a piperazino group which is substituted in the 4-position by a 2-oxo-tetrahydrofuran-4-yl or 2-oxo-tetrahydrofuran-5-ylcarbonyl group,

the tautomers, stereoisomers and the salts thereof.

The following particularly preferred compounds of the above general formula I are mentioned by way of example:

(1) 4-[(3-chloro-4-fluorophenyl)amino]-7-{3-[4-(2-oxo-tetrahydrofuran-4-yl)-piperazin-1-yl]-propyloxy}-6-[(vinylcarbonyl)amino]-quinazoline,

(2) 4-[(3-chloro-4-fluorophenyl)amino]-7-(2-{4-[(S)-(2-oxo-tetrahydrofuran-5-yl)carbonyl]-piperazin-1-yl}-ethoxy)-6-[(vinylcarbonyl)amino]-quinazoline,

(3) 4-[(R)-(1-phenylethyl)amino]-7-{2-[4-(2-oxo-tetra-hydrofuran-4-yl)-piperazin-1-yl]-ethoxy}-6-[(vinylcarbonyl)amino]-quinazoline and

(4) 4-[(3-chloro-4-fluorophenyl)amino]-7-{2-[4-(2-oxo-tetrahydrofuran-4-yl)-piperazin-1-yl]-ethoxy}-6-[(vinylcarbonyl)amino]-quinazoline,

the tautomers, stereoisomers and the salts thereof.

The compounds of general formula I may be prepared by the following methods, for example:

a. reacting a compound of general formula

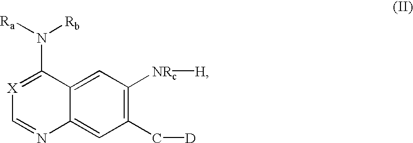

wherein

Rₐ to R_{c}, C, D and X are as hereinbefore defined, with a compound of general formula

Z₁—CO—A—B, (III)

wherein

A and B are as hereinbefore defined and

Z₁ denotes a leaving group such as a halogen atom, e.g. a chlorine or bromine atom, or a hydroxy group.

The reaction is optionally carried out in a solvent or mixture of solvents such as methylene chloride, dimethylformamide, acetonitrile, toluene, chlorobenzene, tetrahydrofuran, methylene chloride/tetrahydrofuran or dioxane, optionally in the presence of an inorganic or organic base and optionally in the presence of a dehydrating agent conveniently at temperatures between −80 and 150° C., preferably at temperatures between −60 and 80° C.

With a compound of general formula III wherein Z₁ denotes a leaving group, the reaction is optionally carried out in a solvent or mixture of solvents such as methylene chloride, dimethylformamide, acetonitrile, toluene, chlorobenzene, tetrahydrofuran, methylene chloride/tetrahydrofuran or dioxane, conveniently in the presence of a tertiary organic base such as triethylamine, pyridine, 2-dimethylaminopyridine or N-ethyl-diisopropylamine (Hünig's base), whilst these organic bases may simultaneously serve as the solvent, or in the presence of an inorganic base such as sodium carbonate, potassium carbonate or sodium hydroxide solution, conveniently at temperatures between −80 and 150° C., preferably at temperatures between −60 and 80° C.

With a compound of general formula III wherein Z₁ denotes a hydroxy group, the reaction is preferably carried out in the presence of a dehydrating agent, e.g. in the presence of isobutyl chloroformate, thionyl chloride, trimethyl chlorosilane, phosphorus trichloride, phosphorus pentoxide, hexamethyldisilazane, N,N′-dicyclohexylcarbodiimide, N,N′-dicyclohexylcarbodiimide/N-hydroxysuccinimide or 1-hydroxy-benzotriazole, and optionally additionally in the presence of 4-dimethylaminopyridine, N,N′-carbonyldiimidazole or triphenylphosphine/carbon tetrachloride, conveniently in a solvent such as methylene chloride, tetrahydrofuran, dioxane, toluene, chlorobenzene, dimethylsulphoxide, ethylene glycol diethyl ether or sulpholane and optionally in the presence of a reaction accelerator such as 4-dimethylaminopyridine at temperatures between −80 and 150° C., but preferably at temperatures between −60 and 80° C.

However, it is particularly advantageous to carry out the reaction with acrylic acid and acrylic acid chloride in the presence of triethylamine.

In the reactions described hereinbefore, any reactive groups present such as hydroxy, carboxy or imino groups may be protected during the reaction by conventional protecting groups which are cleaved again after the reaction.

For example, a protecting group for a hydroxy group may be a trimethylsilyl, acetyl, benzoyl, methyl, ethyl, tert-butyl, trityl, benzyl or tetrahydropyranyl group, protecting groups for a carboxy group may be a trimethylsilyl, methyl, ethyl, tert-butyl, benzyl or tetrahydropyranyl group,

and

protecting groups for an imino group may be a formyl, acetyl, trifluoroacetyl, ethoxycarbonyl, tert-butoxycarbonyl, benzyloxycarbonyl, benzyl, methoxybenzyl or 2,4-dimethoxybenzyl group.

Any protecting group used is optionally subsequently cleaved for example by hydrolysis in an aqueous solvent, e.g. in water, isopropanol/water, acetic acid/water, tetrahydrofuran/water or dioxane/water, in the presence of an acid such as trifluoroacetic acid, hydrochloric acid or sulphuric acid or in the presence of an alkali metal base such as sodium hydroxide or potassium hydroxide or aprotically, e.g. in the presence of iodotrimethylsilane, at temperatures between 0 and 120° C., preferably at temperatures between 10 and 100° C.

However, a benzyl, methoxybenzyl or benzyloxycarbonyl group is cleaved, for example hydrogenolytically, e.g. with hydrogen in the presence of a catalyst such as palladium/charcoal in a suitable solvent such as methanol, ethanol, ethyl acetate or glacial acetic acid, optionally with the addition of an acid such as hydrochloric acid at temperatures between 0 and 100° C., but preferably at room temperatures between 20 and 60° C., and at a hydrogen pressure of 1 to 7 bar, but preferably 3 to 5 bar. A 2,4-dimethoxybenzyl group, however, is preferably cleaved in trifluoroacetic acid in the presence of anisole.

A tert-butyl or tert-butyloxycarbonyl group is preferably cleaved by treating with an acid such as trifluoroacetic acid or hydrochloric acid or by treating with iodotrimethylsilane optionally using a solvent such as methylene chloride, dioxane, methanol or diethyl ether. A trifluoroacetyl group is preferably cleaved by treating with an acid such as hydrochloric acid, optionally in the presence of a solvent such as acetic acid at temperatures between 50 and 120° C. or by treating with sodium hydroxide solution optionally in the presence of a solvent such as tetrahydrofuran at temperatures between 0 and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf. Allinger N. L. and Eliel E. L. in “Topics in Stereochemistry”, Vol. 6, Wiley Interscience, 1971) into their optical antipodes and compounds of general formula I with at least 2 asymmetric carbon atoms may be resolved into their diastereomers on the basis of their different physical properties using methods known per se, e.g. by chromatography and/or fractional crystallisation, and, if these compounds are obtained in racemic form, they may subsequently be resolved into the enantiomers as mentioned above.

The enantiomers are preferably separated by column separation on chiral phases or by recrystallisation from an optically active solvent or by reacting with an optically active substance which forms salts or derivatives such as e.g. esters or amides with the racemic compound, particularly acids and the activated derivatives or alcohols thereof, and separating the diastereomeric mixture of salts or derivatives thus obtained, e.g. on the basis of their differences in solubility, whilst the free antipodes may be released from the pure diastereomeric salts or derivatives by the action of suitable agents. Optically active acids in common use are e.g. the D- and L-forms of tartaric acid or dibenzoyltartaric acid, di-o-tolyltartaric acid, malic acid, mandelic acid, camphorsulphonic acid, glutamic acid, aspartic acid or quinic acid. An optically active alcohol may be for example (+) or (−)-menthol and an optically active acyl group in amides, for example, may be a (+)- or (−)-menthyloxycarbonyl.

Furthermore, the compounds of formula I obtained may be converted into the salts thereof, particularly for pharmaceutical use into the physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

The compounds of general formulae II to III used as starting materials are known from the literature in some cases or may be obtained by methods known from the literature (cf. Examples I to IX).

As already mentioned hereinbefore, the compounds of general formula I according to the invention and the physiologically acceptable salts thereof have valuable pharmacological properties, particularly an inhibiting effect on signal transduction mediated by the Epidermal Growth Factor receptor (EGF-R), whilst this may be achieved for example by inhibiting ligand bonding, receptor dimerisation or tyrosine kinase itself. It is also possible that the transmission of signals to components located downstream is blocked.

The biological properties of the new compounds were investigated as follows:

The inhibition of the EGF-R-mediated signal transmission can be demonstrated e.g. with cells which express human EGF-R and whose survival and proliferation depend on stimulation by EGF or TGF-alpha. A cell line of murine origin dependent on interleukin-3-(IL-3) which was genetically modified to express functional human EGF-R was used here. The proliferation of these cells known as F/L-HERc can therefore be stimulated either by murine IL-3 or by EGF (cf. von Rüden, T. et al. in EMBO J. 7, 2749-2756 (1988) and Pierce, J. H. et al. in Science 239, 628-631 (1988)).

The starting material used for the F/L-HERc cells was the cell line FDC-P₁, the production of which has been described by Dexter, T. M. et al. in J. Exp. Med. 152, 1036-1047 (1980). Alternatively, however, other growth-factor-dependent cells may also be used (cf. for example Pierce, J. H. et al. in Science 239, 628-631 (1988), Shibuya, H. et al. in Cell 70, 57-67 (1992) and Alexander, W. S. et al. in EMBO J. 10, 3683-3691 (1991)). For expressing the human EGF-R cDNA (cf. Ullrich, A. et al. in Nature 309, 418-425 (1984)) recombinant retroviruses were used as described by von Rüden, T. et al., EMBO J. 7, 2749-2756 (1988), except that the retroviral vector LXSN (cf. Miller, A. D. et al. in BioTechniques 7, 980-990 (1989)) was used for the expression of the EGF-R cDNA and the line GP+E86 (cf. Markowitz, D. et al. in J. Virol. 62, 1120-1124 (1988)) was used as the packaging cell.

The test was performed as follows:

F/L-HERc cells were cultivated in RPMI/1640 medium (BioWhittaker), supplemented with 10% foetal calf serum (FCS, Boehringer Mannheim), 2 mM glutamine (BioWhittaker), standard antibiotics and 20 ng/ml of human EGF (Promega), at 37° C. and 5% CO₂. In order to investigate the inhibitory activity of the compounds according to the invention, 1.5×10⁴ cells per well were cultivated in triplicate in 96-well dishes in the above medium (200 μl), the cell proliferation being stimulated with either EGF (20 ng/ml) or murine IL-3. The IL-3 used was obtained from culture supernatants of the cell line X63/0 mIL-3 (cf. Karasuyama, H. et al. in Eur. J. Immunol. 18, 97-104 (1988)). The compounds according to the invention were dissolved in 100% dimethylsulphoxide (DMSO) and added to the cultures in various dilutions, the maximum DMSO concentration being 1%. The cultures were incubated for 48 hours at 37° C.

In order to determine the inhibitory activity of the compounds according to the invention the relative cell number was measured in O.D. units using the Cell Titer 96™ AQᵤₑₒᵤₛ Non-Radioactive Cell Proliferation Assay (Promega). The relative cell number was calculated as a percentage of the control (F/LHERc cells without inhibitor) and the concentration of active substance which inhibits the proliferation of the cells by 50% (IC₅₀) was derived therefrom. The following results were obtained:

| | Inhibition of EGF- |
|---|---|
| Compound | dependent proliferation |
| (Example No.) | IC₅₀ [nM] |
| 1 (2) | 12 |

The compounds of general formula I according to the invention thus inhibit the signal transduction by tyrosine kinases, as demonstrated by the example of the human EGF receptor, and are therefore useful for treating pathophysiological processes caused by hyperfunction of tyrosine kinases. These are e.g. benign or malignant tumours, particularly tumours of epithelial and neuroepithelial origin, metastasisation and the abnormal proliferation of vascular endothelial cells (neoangiogenesis).

The compounds according to the invention are also useful for preventing and treating diseases of the airways and lungs which are accompanied by increased or altered production of mucus caused by stimulation of tyrosine kinases, e.g. in inflammatory diseases of the airways such as chronic bronchitis, chronic obstructive bronchitis, asthma, bronchiectasias, allergic or non-allergic rhinitis or sinusitis, cystic fibrosis, α1-antitrypsin deficiency, or coughs, pulmonary emphysema, pulmonary fibrosis and hyperreactive airways.

The compounds are also suitable for treating diseases of the gastrointestinal tract and bile duct and gall bladder which are associated with disrupted activity of the tyrosine kinases, such as may be found e.g. in chronic inflammatory changes such as cholecystitis, Crohn's disease, ulcerative colitis, and ulcers in the gastrointestinal tract or such as may occur in diseases of the gastrointestinal tract which are associated with increased secretions, such as Menetrier's disease, secreting adenomas and protein loss syndrome, also for treating nasal polyps and polyps of the gastrointestinal tract of various origins, such as for example villous or adenomatous polyps of the large bowel, but also polyps in familial polyposis coli, intestinal polyps in Gardner's syndrome, polyps throughout the entire gastrointestinal tract in Peutz-Jeghers Syndrome, inflammatory pseudopolyps, juvenile polyps, colitis cystica profunda and pneumatosis cystoides intestinales.

Moreover, the compounds of general formula I and the physiologically acceptable salts thereof may be used to treat kidney diseases, particularly cystic changes as in cystic kidneys, for treating renal cysts which may be idiopathic in origin or which occur in syndromes such as e.g. tubercular sclerosis, in von-Hippel-Lindau Syndrome, in nephronophthisis and spongy kidney and other diseases caused by abnormal functioning of tyrosine kinases such as e.g. epidermal hyperproliferation (psoriasis), inflammatory processes, diseases of the immune system, hyperproliferation of haematopoietic cells, etc.

By reason of their biological properties the compounds according to the invention may be used on their own or in conjunction with other pharmacologically active compounds, for example in tumour therapy, in monotherapy or in conjunction with other anti-tumour therapeutic agents, for example in combination with topoisomerase inhibitors (e.g. etoposide), mitosis inhibitors (e.g. vinblastin), compounds which interact with nucleic acids (e.g. cis-platin, cyclophosphamide, adriamycin), hormone antagonists (e.g. tamoxifen), inhibitors of metabolic processes (e.g. 5-FU etc.), cytokines (e.g. interferons), antibodies, etc. For treating respiratory tract diseases, these compounds may be used on their own or in conjunction with other therapeutic agents for the airways, such as substances with a secretolytic, broncholytic and/or anti-inflammatory activity. For treating diseases in the region of the gastrointestinal tract, these compounds may also be administered on their own or in conjunction with substances having an effect on motility or secretion or with anti-inflammatory substances. These combinations may be administered either simultaneously or sequentially.

These compounds may be administered either on their own or in conjunction with other active substances by intravenous, subcutaneous, intramuscular, intrarectal, intraperitoneal or intranasal route, by inhalation or transdermally or orally, whilst aerosol formulations are particularly suitable for inhalation.

For pharmaceutical use the compounds according to the invention are generally used for warm-blooded vertebrates, particularly humans, in doses of 0.01-100 mg/kg of body weight, preferably 0.1-15 mg/kg. For administration they are formulated with one or more conventional inert carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethyleneglycol, propyleneglycol, stearylalcohol, carboxymethylcellulose or fatty substances such as hard fat or suitable mixtures thereof in conventional galenic preparations such as plain or coated tablets, capsules, powders, suspensions, solutions, sprays or suppositories.

The following Examples are intended to illustrate the present invention without restricting it:

Preparation of the starting compounds:

### EXAMPLE I

6-amino-4-[(3-chloro-4-fluorophenyl)amino]-7-{3-[4-(2-oxo-tetrahydrofuran-4-yl)-piperazin-1-yl]-propyloxy}-quinazoline

610 mg of 4-[(3-chloro-4-fluorophenyl)amino]-6-nitro-7-{3-[4-(2-oxo-tetrahydrofuran-4-yl)-piperazin-1-yl]-propyloxy}-quinazoline and 268 mg iron powder are suspended in 22 ml of ethanol and heated to boiling. Then 0.76 ml of glacial acetic acid and 0.50 ml of water are added. Within a few minutes a clear brown solution has formed and after one hour the reduction is finished. For working up, the reaction mixture is evaporated down. The residue is stirred with methylene chloride, mixed with a few lumps of ice and made alkaline with 1 ml of 1 SN sodium hydroxide solution. The aqueous phase is separated off and extracted with methylene chloride/methanol (95:5). The combined organic phases are washed with water, dried over magnesium sulphate and evaporated down. The resin-like residue is crystallised by stirring with tert-butyl methyl ether. The yellowish solid is suction filtered and dried in vacuo.

Yield: 437 mg (76% of theoretical),

R_{f} value: 0.30 (silica gel, methylene chloride/methanol/concentrated aqueous ammonia solution=90:10:0.1)

Mass spectrum (ESI⁺): m/z=515, 517 [M+H]⁺

The following compounds are obtained analogously to Example I:

(1) 6-amino-4-[(3-chloro-4-fluorophenyl)amino]-7-(2-{4-[(S)-(2-oxo-tetrahydrofuran-5-yl)carbonyl]-piperazin-1-yl}-ethoxy)-quinazoline

R_{f} value: 0.38 (silica gel, methylene chloride/methanol/concentrated aqueous ammonia solution=90:10:0.1)

Mass spectrum (ESI+): m/z=529, 531 [M+H]⁺

(2) 6-amino-4-[(R)-(1-phenylethyl)amino]-7-{2-[4-(2-oxo-tetrahydrofuran-4-yl)-piperazin-1-yl]-ethoxy}-quinazoline

R_{f} value: 0.36 (silica gel, methylene chloride/methanol/concentrated aqueous ammonia solution=90:10:1)

Mass spectrum (ESI⁺): m/z=477 [M+H]⁺

(3) 6-amino-4-[(3-chloro-4-fluorophenyl)amino]-7-{2-[4-(2-oxo-tetrahydrofuran-4-yl)-piperazin-1-yl]-ethoxy}-quinazoline

R_{f} value: 0.29 (silica gel, methylene chloride/methanol/concentrated aqueous ammonia solution=90:10:1)

Mass spectrum (ESI⁺): m/z=501, 503 [M+H]⁺

### EXAMPLE II

4-[(3-chloro-4-fluorophenyl)amino]-6-nitro-7-{3-[4-(2-oxo-tetrahydrofuran-4-yl)-piperazin-1-yl]-propyloxy}-quinazoline

1.10 g of 4-[(3-chloro-4-fluorophenyl)amino]-7-(3-methanesulphonyloxy-propyloxy)-6-nitro-quinazoline and 2.33 g of 4-piperazin-1-yl-dihydrofuran-2-one x 2 trifluoroacetic acid in 25 ml of acetonitrile are combined with 360 mg of sodium iodide and 1.63 g of potassium carbonate. The reaction mixture is refluxed for about two hours. For working up, the inorganic salts are filtered off and washed with ethyl acetate and methylene chloride/methanol. The filtrate is evaporated down and the evaporation residue is taken up in methylene chloride/methanol. The solution is washed with water, dried over magnesium sulphate and evaporated down. The yellow, resin-like residue is chromatographed using a silica gel column with methylene chloride/methanol/concentrated aqueous ammonia solution (95:4:1). The title compound is obtained as a yellow solid.

Yield: 625 mg (49% of theoretical),

R_{f} value: 0.45 (silica gel, methylene chloride/methanol/concentrated aqueous ammonia solution=90:10:0.1)

Mass spectrum (ESI⁺): m/z=545, 547 [M+H]⁺

The following compounds are obtained analogously to Example II:

(1) 4-[(3-chloro-4-fluorophenyl)amino]-7-{2-[4-(tert-butyloxycarbonyl)-piperazin-1-yl]-ethoxy}-6-nitro-quinazoline

R_{f} value: 0.42 (silica gel, methylene chloride/methanol/concentrated aqueous ammonia solution=90:10:1)

(2) 4-[(R)-(1-phenylethyl)amino]-7-{2-[4-(tert-butyloxycarbonyl)-piperazin-1-yl]-ethoxy}-6-nitro-quinazoline

R_{f} value: 0.20 (silica gel, methylene chloride/methanol 95:5)

Mass spectrum (ESI⁻): m/z=521 [M-H]⁻

(3) 4-[(3-chloro-4-fluorophenyl)amino]-7-{2-[4-(2-oxo-tetrahydrofuran-4-yl)-piperazin-1-yl]-ethoxy}-6-nitro-quinazoline

R_{f} value: 0.43 (silica gel, methylene chloride/methanol/concentrated aqueous ammonia solution=90:10:0.1)

Mass spectrum (ESI⁻): m/z=529, 531 [M-H]⁻

(4) 4-[(3-chloro-4-fluorophenyl)amino]-7-{3-[4-(tert.-butyloxycarbonyl)-piperazin-1-yl]-propyloxy}-6-nitro-quinazoline

R_{f} value: 0.55 (silica gel, methylene chloride/methanol=9:1)

Mass spectrum (ESI⁺): m/z=561, 563 [M+H]⁺

(5) 4-[(3- chloro-4-fluorophenyl)amino]-7-{4-[4-(tert.-butyloxycarbonyl)-piperazin-1-yl]-butyloxy}-6-nitro- quinazoline

R_{f} value: 0.49 (silica gel, methylene chloride/methanol=9:1)

Mass spectrum (ESI⁺): m/z=597, 599 [M+Na]⁺

### EXAMPLE III

4-[(3-chloro-4-fluorophenyl)amino] -7-(3-methanesulphonyloxy-propyloxy)-6-nitro-quinazoline

0.96 ml of methanesulphonic acid chloride are added dropwise, with stirring, to 4.60 g of 4-[(3-chloro-4-fluorophenyl)-amino] -7-(3-hydroxy-propyloxy)-6-nitro-quinazoline and 4.29 ml of diisopropylethylamine in 150 ml methylene chloride at ambient temperature. The reaction mixture is stirred for about 30 minutes at ambient temperature, then another 0.1 ml of methanesulphonic acid chloride are added. After about one hour the reaction is complete and the cloudy reaction solution is mixed with ice water. A thick, yellowish precipitate is formed which is suction filtered, washed with a little methylene chloride and water and dried in the desiccator.

Yield: 5.06 g (92% of theoretical),

R_{f} value: 0.43 (silica gel, methylene chloride/methanol=95:5)

Mass spectrum (ESI⁻): m/z=469, 471 [M-H]⁻

The following compounds are obtained analogously to Example III:

(1) 4-[(3-chloro-4-fluorophenyl)amino]-7-(2-methanesulphonyloxy-ethoxy)-6-nitro-quinazoline

R_{f} value: 0.53 (silica gel, methylene chloride/methanol/concentrated aqueous ammonia solution=90:10:1)

Mass spectrum (ESI⁻): m/z=455, 457 [M-H]⁻

(2) 4-[(R)-(1-phenylethyl)amino]-7-(2-methanesulphonyloxy-ethoxy)-6-nitro-quinazoline

R_{f} value: 0.45 (silica gel, methylene chloride/methanol=95:5) Mass spectrum (ESI⁻): m/z=431 [M-H]⁻

(3) 4-[(3-chloro-4-fluorophenyl)amino]-7-(4-methanesulphonyloxy-butyloxy)-6-nitro-quinazoline

R_{f} value: 0.42 (silica gel, methylene chloride/methanol=95:5)

Mass spectrum (ESI⁻): m/z=483, 485 [M-H]⁻

### EXAMPLE IV

4-[(3-chloro-4-fluorophenyl)amino]-7-(3-hydroxy-propyloxy)-6-nitro-quinazoline

3.00 ml of concentrated hydrochloric acid are added dropwise to 21.30 g of 4-[(3-chloro-4-fluorophenyl)amino]-7-[3-(tetrahydropyran-2-yloxy)-propyloxy]-6-nitro-quinazoline (crude product from Example V) in 200 ml methanol. A yellow precipitate is formed. The suspension is stirred for another 3.5 hours at 50° C. For working up the methanol is distilled off in vacuo using a rotary evaporator. The residue is combined with ethyl acetate and some ice water and made alkaline with sodium hydroxide solution. The organic phase is washed with water and saturated sodium chloride solution and left to stand overnight at ambient temperature, during which time a yellow precipitate is formed. This is suction filtered, washed with ethyl acetate and dried. The filtrate is evaporated down and the evaporation residue is recrystallised from ethyl acetate. The crystals thus obtained are combined with the precipitate previously suction filtered and again recrystallised from ethyl acetate. The desired product is obtained in the form of slightly yellowish crystals.

Yield: 4.60 g (40% of theoretical),

Melting point: 224-227° C.

Mass spectrum (ESI⁻): m/z=391, 393 [M-H]⁻

The following compounds are obtained analogously to Example IV:

(1) 4-[(3-chloro-4-fluorophenyl)amino]-7-(2-hydroxy-ethoxy)-6-nitro-quinazoline

R_{f} value: 0.46 (silica gel, methylene chloride/methanol/concentrated aqueous ammonia solution=90:10:1)

Mass spectrum (ESI⁻): m/z=377, 379 [M-H]⁻

(2) 4-[(R)-(1-phenyl-ethyl)amino]-7-(2-hydroxy-ethoxy)-6-nitro-quinazoline

Melting point: 192-194° C.

Mass spectrum (ESI⁻): m/z=353 [M-H]⁻

(3) 4-[(3-chloro-4-fluorophenyl)amino]-7-(4-hydroxy-butyloxy)-6-nitro-quinazoline

R_{f} value: 0.25 (silica gel, methylene chloride/methanol=95:5)

Mass spectrum (ESI⁻): m/z=405, 407 [M-H]⁻

### EXAMPLE V

4-[(3-chloro-4-fluorophenyl)amino]-7-[3-(tetrahydropyran-2-yloxy)-propyloxy]-6-nitro-quinazoline

2.40 g of sodium hydride (60% in mineral oil) are added batchwise to 14.50 g of 3-(tetrahydropyran-2-yloxy)-propan-1-ol in 120 ml tetrahydrofuran. The reaction mixture is stirred for about 15 minutes at ambient temperature, then 10.10 g of 4-[(3-chloro-4-fluorophenyl)amino]-7-fluoro-6-nitro-quinazoline are added while cooling with an ice bath and rinsed with 20 ml of tetrahydrofuran. The reaction mixture suddenly turns dark red and the ice bath is removed. After about 2.5 hours a total of 500 mg of sodium hydride are added in two batches and the reaction mixture is stirred overnight at ambient temperature. For working up, the dark reaction solution is poured onto about 400 ml of ice water, mixed with tert-butyl methyl ether and ethyl acetate and neutralised with citric acid. The organic phase is separated off and evaporated down. 21.30 g of a brown oil are obtained, which is subjected to cleavage of the protecting groups without any further purification (cf. Example IV).

R_{f} value: 0.37 (silica gel, cyclohexane/ethyl acetate=1:1)

Mass spectrum (ESI⁻): m/z=475, 477 [M-H]⁻

The following compounds are obtained analogously to Example V:

(1) 4-[(3-chloro-4-fluorophenyl)amino]-7-[2-(tetrahydropyran-2-yloxy)-ethoxy]-6-nitro-quinazoline

R_{f} value: 0.60 (silica gel, petroleum ether/ethyl acetate=1:2)

Mass spectrum (ESI⁻): m/z=461, 463 [M-H]⁻

(2) 4-[(R)-(1-phenyl-ethyl)amino]-7-[2-(tetrahydropyran-2-yloxy)-ethoxy]-6-nitro-quinazoline

R_{f} value: 0.12 (silica gel, cyclohexane/ethyl acetate=1:1)

Mass spectrum (ESI⁻): m/z=437 [M-H]⁻

(3) 4-[(3-chloro-4-fluorophenyl)amino]-7-[4-(tetrahydropyran-2-yloxy)-butyloxy]-6-nitro-quinazoline

R_{f} value: 0.31 (silica gel, cyclohexane/ethyl acetate=1:1)

Mass spectrum (ESI⁻): m/z=489, 491 [M-H]⁻

### EXAMPLE VI

4-[(3-chloro-4-fluorophenyl)amino]-7-(2-{4-[(S)-(2-oxo-tetrahydrofuran-5-yl)carbonyl]-piperazin-1-yl}-ethoxy)-6-nitro-quinazoline

93 mg of (S)-(+)-5-oxo-tetrahydrofuran-2-carboxylic acid and 176 μl of triethylamine are added to 320 mg of 4-[(3-chloro-4-fluorophenyl)amino]-7-[2-(piperazin-1-yl)-ethoxy]-6-nitro-quinazoline in 4 ml of N,N-dimethylformamide. Then the reaction mixture is combined with 230 mg of (benzotriazol-1-yl)-N,N,N′,N′-tetramethyl-uronium-tetrafluoroborate and stirred for four hours at ambient temperature. For working up, about 20 ml of ice water are added. The precipitate formed is suction filtered, washed with water and tert-butyl methyl ether and dried in the desiccator. The ochre-coloured solid crude product is further reacted without further purification. Yield: 330 mg (82% of theoretical), R_{f} value: 0.40 (silica gel, methylene chloride/methanol/concentrated aqueous ammonia solution=90:10:0.1)

The following compounds are obtained analogously to Example VI:

(1) 4-[(3-chloro-4-fluorophenyl)amino]-7-(3-{4-[(S)-(2-oxo-tetrahydrofuran-5-yl)carbonyl]-piperazin-1-yl}-propyloxy)-6-nitro-quinazoline

R_{f} value: 0.48 (silica gel, methylene chloride/methanol/conc. aqueous ammonia solution=90:10:0.1)

Mass spectrum (ESI⁺): m/z=573, 575 [M+H]⁺

(2) 4-[(3-chloro-4-fluorophenyl)amino]-7-(4-{4-[(S)-(2-oxo-tetrahydrofuran-5-yl)carbonyl]-piperazin-1-yl}-butyloxy)-6-nitro-quinazoline

R_{f} value: 0.47 (silica gel, methylene chloride/methanol/conc. aqueous ammonia solution=90:10:0.1)

Mass spectrum (ESI⁻): m/z=585, 587 [M-H]⁻

### EXAMPLE VII

4-[(3-chloro-4-fluorophenyl)amino]-7-[2-(piperazin-1-yl)-ethoxy]-6-nitro-quinazoline

780 mg of 4-[(3-chloro-4-fluorophenyl)amino]-7-{2-[4-(tert-butyloxycarbonyl)-piperazin-1-yl]-ethoxy}-6-nitro-quinazoline in 10 ml of methylene chloride are combined with 2.00 ml of trifluoroacetic acid. The yellow reaction solution is stirred for one hour at ambient temperature and then left to stand overnight. The next morning, the reaction mixture is evaporated down, mixed with about 20 ml of water and made alkaline with concentrated ammonia solution. The precipitate formed is suction filtered and washed with water and tert-butyl methyl ether. The yellowish solid is taken up in methylene chloride/methanol (5:1). The solution is washed with 2 N sodium hydroxide solution. The aqueous phase is extracted with a total of 400 ml of methylene chloride/methanol (5:1). The combined organic phases are washed with saturated sodium chloride solution, dried over magnesium sulphate and evaporated down. The flask residue is triturated with tert-butyl methyl ether, suction filtered and dried in a desiccator.

Yield: 680 mg (5% of theoretical),

R_{f} value: 0.15 (silica gel, methylene chloride/methanol/concentrated aqueous ammonia solution=90:10:1)

Mass spectrum (ESI⁻): m/z=445, 447 [M-H]⁻

The following compounds are obtained analogously to Example VII:

(1) 4-[(R)-(1-phenyl-ethyl)amino]-7-[2-(piperazin-1-yl)-ethoxy]-6-nitro-quinazoline

R_{f} value: 0.12 (silica gel, methylene chloride/methanol/concentrated aqueous ammonia solution=90:10:1)

Mass spectrum (ESI⁻): m/z=421 [M-H]⁻

(2) 4-(piperazin-1-yl)-dihydrofuran-2-one x 2 trifluoroacetic acid (The reaction solution is evaporated down without any aqueous working up.)

R_{f} value: 0.09 (silica gel, methylene chloride/methanol/concentrated aqueous ammonia solution=90:10:1)

Mass spectrum (ESI⁺): m/z=171 [M+H]⁺

(3) 4-[(3-chloro-4-fluorophenyl)amino]-7-[3-(piperazin-1-yl)-propyloxy]-6-nitro-quinazoline

R_{f} value: 0.18 (silica gel, methylene chloride/methanol/concentrated aqueous ammonia solution=90:10:1)

Mass spectrum (ESI⁺): m/z=461, 463 [M+H]⁺

(4) 4-[(3-chloro-4-fluorophenyl)amino]-7-[4-(piperazin-1-yl)-butyloxy]-6-nitro-quinazoline

R_{f} value: 0.20 (silica gel, methylene chloride/methanol/concentrated aqueous ammonia solution=90:10:1)

Mass spectrum (ESI⁺): m/z=475, 477 [M+H]⁺

### EXAMPLE VIII

4-[(R)-(1-phenylethyl)amino]-7-{2-[4-(2-oxo-tetrahydrofuran-4-yl)-piperazin-1-yl]-ethoxy}-6-nitro-quinazoline

1.99 g of 4-[(R)-(1-phenyl-ethyl)amino]-7-[2-(piperazin-1-yl)-ethoxy]-6-nitro-quinazoline are dissolved in 10 ml of methanol and combined with 376 μl of (5H)-furan-2-one. The reaction mixture is stirred overnight at ambient temperature, then another 35 μl of (5H)-furan-2-one are added. After another 1.5 hours' stirring at ambient temperature the reaction is complete. The brown reaction solution is evaporated down and chromatographed using a silica gel column, with methylene chloride/methanol (95:5 to 93:7) as eluant. The title compound is obtained as a yellowish solid.

Yield: 1.71 g (72% of theoretical),

R_{f} value: 0.45 (silica gel, methylene chloride/methanol/concentrated aqueous ammonia solution 90:10:1)

Mass spectrum (ESI⁻): m/z 505 [M-H]⁻

The following compound is obtained analogously to Example VIII:

(1) 4-(4-tert-butyloxy-piperazin-1-yl)-dihydrofuran-2-one (The reaction is carried out in methylene chloride.)

R_{f} value: 0.54 (silica gel, methylene chloride/methanol/concentrated aqueous ammonia solution=90:10:1)

Mass spectrum (ESI⁺): m/z=293 [M+Na]⁺

(2) 4-[(3-chloro-4-fluorophenyl)amino]-7-{4-[4-(2-oxo-tetrahydrofuran-4-yl)-piperazin-1-yl]-butyloxy}-6-nitro-quinazoline

R_{f} value: 0.50 (silica gel, methylene chloride/methanol/concentrated aqueous ammonia solution=90:10:0.1)

Mass spectrum (ESI⁺): m/z=559, 561 [M+H]⁺

### EXAMPLE IX

4-[(R)-(1-phenyl-ethyl)amino]-6-nitro-7-fluoro-quinazoline

A solution of 74 ml of (R)-1-phenyl-ethylamine in 100 ml of dioxane is added dropwise, while cooling with an ice bath, to 108.8 g of 4-chloro-6-nitro-7-fluoro-quinazoline in 800 ml methylene chloride. The reaction mixture is stirred overnight at ambient temperature. For working up it is extracted with water. The organic phase is dried over magnesium sulphate and evaporated down. The residue is purified by chromatography using a silica gel column with petroleum ether/ethyl acetate (1:1) as eluant.

Yield 52.90 g (35% of theoretical),

Melting point: 203° C.

Mass spectrum (ESI⁺): m/z=313 [M+H]⁺

### EXAMPLE X

6-Amino-4-[(3-chloro-4-fluorophenyl)amino]-7-(3-{4-[(S)-(2-oxo-tetrahydrofuran-5-yl)carbonyl]-piperazin-1-yl}-propyloxy)-quinazoline The substance is obtained in a 75% yield by hydrogenation of 4-[(3-chloro-4-fluorophenyl)amino]-7-(3-{4-[(S)-(2-oxo-tetrahydrofuran-5-yl)carbonyl]-piperazin-1-yl}-propyloxy)-6-nitro-quinazoline in tetrahydrofuran in the presence of Raney nickel in a Parr apparatus at a partial hydrogen pressure of 50 psi.

R_{f} value: 0.44 (silica gel, methylene chloride/methanol/concentrated aqueous ammonia solution=90:10:0.1)

Mass spectrum (ESI⁻): m/z=541, 543 [M-H]⁻

The following compounds are obtained analogously to Example X:

(1) 6-Amino-4-[(3-chloro-4-fluorophenyl)amino]-7-{4-[4-(2-oxo-tetrahydrofuran-4-yl)-piperazin-1-yl]-butyloxy}-quinazoline

R_{f} value: 0.24 (silica gel, methylene chloride/methanol/concentrated aqueous ammonia solution=90:10:0.1)

Mass spectrum (ESI⁺): m/z=529, 531 [M+H]⁺

(2) 6-Amino-4-[(3-chloro-4-fluorophenyl)amino]-7-(4-{4-[(S)-(2-oxo-tetrahydrofuran-5-yl)carbonyl]-piperazin-1-yl}-butyloxy)-quinazoline

R_{f} value: 0.35 (silica gel, methylene chloride/methanol/concentrated aqueous ammonia solution=90:10:0.1)

Mass spectrum (ESI⁺): m/z=579, 581 [M+Na]⁺

Preparation of the final compounds:

### EXAMPLE 1

4-[(3-chloro-4-fluorophenyl)amino]-7-{3-[4-(2-oxo-tetrahydrofuran-4-yl)-piperazin-1-yl]-propyloxy}-6-[(vinylcarbonyl)amino]-quinazoline

A mixture of 166 mg of acrylic acid and 0.77 ml of triethylamine in 10 ml of tetrahydrofuran is cooled to −50° C. in a dry ice/acetone cooling bath and mixed with a solution of 175 μl of acrylic acid chloride in 4 ml of tetrahydrofuran. The reaction mixture is stirred for 45 minutes at this temperature. Then a solution of 427 mg of 6-amino-4-[(3-chloro-4-fluorophenyl)amino]-7-{3-[4-(2-oxo-tetrahydrofuran-4-yl)-piperazin-1-yl]-propyloxy}-quinazoline in 10 ml of tetrahydrofuran is added within 20 minutes. The reaction mixture is then left to come up slowly to 0° C. and stirred at this temperature until the reaction is complete. Ice water is then added, whereupon a viscous precipitate is formed. This is thoroughly extracted several times with ethyl acetate/methanol. The combined organic phases are washed with saturated sodium chloride solution, dried over magnesium sulphate and evaporated down. The yellowish, resin-like crude product is purified by chromatography using a silica gel column with methylene chloride/methanol (95:5) as eluant.

Yield: 148 mg (31% of theoretical),

R_{f} value: 0.45 (silica gel, methylene chloride/methanol/concentrated aqueous ammonia solution=90:10:0.1)

Mass spectrum (ESI⁻): m/z=567, 569 [M-H]⁻

The following compounds are obtained analogously to Example 1:

(1) 4-[(3-chloro-4-fluorophenyl)amino]-7-(2-{4-[(S)-(2-oxo-tetrahydrofuran-5-yl)carbonyl]-piperazin-1-yl}-ethoxy)-6-[(vinylcarbonyl)amino]-quinazoline

R_{f} value: 0.46 (silica gel, methylene chloride/methanol/concentrated aqueous ammonia solution=90:10:0.1)

Mass spectrum (ESI⁻): m/z=581, 583 [M-H]⁻

(2) 4-[(R)-(1-phenylethyl)amino]-7-{2-[4-(2-oxo-tetrahydrofuran-4-yl)-piperazin-1-yl]-ethoxy}-6-[(vinylcarbonyl)amino]-quinazoline (The reaction is carried out only with acrylic acid chloride in methylene chloride in the presence of triethylamine.)

R_{f} value: 0.42 (silica gel, methylene chloride/methanol/concentrated aqueous ammonia solution=90:10:1)

Mass spectrum (ESI⁻): m/z=529 [M-H]⁻

(3) 4-[(3-chloro-4-fluorophenyl)amino]-7-{2-[4-(2-oxo-tetrahydrofuran-4-yl)-piperazin-1-yl]-ethoxy}-6-[(vinylcarbonyl)amino]-quinazoline (The reaction is carried out with acrylic acid and isobutyl chloroformate in the presence of triethylamine in tetrahydrofuran.)

R_{f} value: 0.40 (silica gel, methylene chloride/methanol/concentrated aqueous ammonia solution=90:10:1)

Mass spectrum (ESI⁻): m/z=553, 555 [M-H]⁻

(4) 4-[(3-chloro-4-fluorophenyl)amino]-7-(3-{4-[(S)-(2-oxo-tetrahydrofuran-5-yl)carbonyl]-piperazin-1-yl}-propyloxy)-6-[(vinylcarbonyl)amino]-quinazoline

R_{f} value: 0.26 (silica gel, methylene chloride/methanol=9:1)

Mass spectrum (ESI⁺): m/z=597, 599 [M+H]⁺

(5) 4-[(3-chloro-4-fluorophenyl)amino]-7-{4-[4-(2-oxo-tetrahydrofuran-4-yl)-piperazin-1-yl]-butyloxy}-6-[(vinylcarbonyl)amino]-quinazoline

R_{f} value: 0.28 (silica gel, methylene chloride/methanol/concentrated aqueous ammonia solution=90:10:0.1)

Mass spectrum (ESI⁺): m/z=583, 585 [M+H]⁺

(6) 4-[(3-chloro-4-fluorophenyl)amino]-7-(4-{4-[(S)-(2-oxo-tetrahydrofuran-5-yl)carbonyl]-piperazin-1-yl}-butyloxy)-6-[(vinylcarbonyl)amino]-quinazoline

R_{f} value: 0.45 (silica gel, methylene chloride/methanol/concentrated aqueous ammonia solution=90:10:0.1)

Mass spectrum (ESI⁺): m/z=611, 613 [M+H]⁺

The following compounds can be prepared analogously to the foregoing Examples and other methods known from the literature:

(1) 4-[(3-chloro-4-fluorophenyl)amino]-7-(3-{4-[(2-oxo-tetrahydrofuran-5-yl)methyl]-piperazin-1-yl}-propyloxy)-6-[(vinylcarbonyl)amino]-quinazoline

(2) 4-[(3-chloro-4-fluorophenyl)amino]-7-[3-(4-{2-[(2-oxo-tetrahydrofuran-3-yl)sulphanyl]-ethyl}-piperazin-1-yl)-propyloxy]-6-[(vinylcarbonyl)amino]-quinazoline

(3) 4-[(3-chloro-4-fluorophenyl)amino]-7-{3-[1-(2-oxo-tetrahydrofuran-4-yl)-piperidin-4-yl]-propyloxy}-6-[(vinylcarbonyl)amino]-quinazoline

(4) 4-[(3-bromophenyl)amino]-7-{3-[1-(2-oxo-tetrahydrofuran-4-yl)-piperidin-4-yl]-propyloxy}-6-[(vinylcarbonyl)amino]-quinazoline

(5) 4-[(3-methylphenyl)amino]-7-{3-[1-(2-oxo-tetrahydrofuran-4-yl)-piperidin-4-yl]-propyloxy}-6-[(vinylcarbonyl)amino]-quinazoline

(6) 4-[(3-chloro-4-fluorophenyl)amino]-7-[3-(3-oxo-perhydro-pyrazino[2, 1-c][1,4]oxazin-8-yl) -propyloxy]-6-[(vinylcarbonyl)amino]-quinazoline

(7) 4-[(3-chloro-4-fluorophenyl)amino]-7-[3-(1-oxo-perhydro-pyrazino[2, 1-c][1,4]oxazin-8-yl) -propyloxy]-6-[(vinylcarbonyl)amino]-quinazoline

(8) 4-[(3-chloro-4-fluorophenyl)amino]-7-[3-(2-oxa-3-oxo-8-aza-spiro[4,5]dec-8-yl)-propyloxy]-6-[(vinylcarbonyl)amino]-quinazoline

(9) 4-[(3-chloro-4-fluorophenyl)amino]-7-[3-(3-oxa-2-oxo-9-aza-spiro[5.5]undecan-9-yl)-propyloxy]-6-[(vinylcarbonyl)amino]-quinazoline

(10) 4-[(3-chloro-4-fluorophenyl)amino]-7-[3-(1,4-dioxa-2-oxo-9-aza-spiro[5.5]undecan-9-yl)-propyloxy]-6-[(vinylcarbonyl)amino]-quinazoline

(11) 4-[(3-chloro-4-fluorophenyl)amino]-7-[3-(4-methyl-1-oxa-2-oxo-4,9-diaza-spiro[5.5]undecan-9-yl)-propyloxy]-6-[(vinylcarbonyl)amino]-quinazoline

(12) 4-[(3-chloro-4-fluorophenyl)amino]-7-{3-[4-(2-oxo-morpholin-4-yl)-piperidin-1-yl]-propyloxy}-6-[(vinylcarbonyl)amino]-quinazoline

(13) 4-[(3-chloro-4-fluorophenyl)amino]-7-{3-[4-(6-methyl-2-oxo-morpholin-4-yl)-piperidin-1-yl]-propyloxy}-6-[(vinylcarbonyl)amino]-quinazoline

(14) 4-[(3-chloro-4-fluorophenyl)amino]-7-(3-{4-[(6-methyl-2-oxo-morpholin-4-yl)methyl]-piperidin-1-yl}-propyloxy)-6-[(vinylcarbonyl)amino]-quinazoline

(15) 4-[(3-chloro-4-fluorophenyl)amino]-7-(3-{4-[(2-oxo-tetrahydrofuran-3-yl)sulphanyl]-piperidin-1-yl}-propyloxy)-6-[(vinylcarbonyl)amino]-quinazoline

(16) 4-[(3-chloro-4-fluorophenyl)amino]-7-[3-(6-methoxymethyl-2-oxo-morpholin-4-yl)-propyloxy]-6-[(vinylcarbonyl)amino] -quinazoline

(17) 4-[(3-chloro-4-fluorophenyl)amino]-7-{3-[6-(2-methoxy-ethyl)-2-oxo-morpholin-4-yl]-propyloxy}-6-[(vinylcarbonyl)amino]-quinazoline

(18) 4-[(3-chloro-4-fluorophenyl)amino]-7-[3-(1,9-dioxa-2-oxo-4-aza-spiro[5.5]undecan-4-yl)-propyloxy]-6-[(vinylcarbonyl)amino]-quinazoline

### EXAMPLE 2

Coated Tablets Containing 75 mg of Active Substance

One tablet core contains:

| | |
|---|---|
| active substance | 75.0 mg |
| calcium phosphate | 93.0 mg |
| corn starch | 35.5 mg |
| polyvinylpyrrolidone | 10.0 mg |
| hydroxypropylmethylcellulose | 15.0 mg |
| magnesium stearate | 1.5 mg |
| | 230.0 mg |

Preparation:

The active substance is mixed with calcium phosphate, corn starch, polyvinylpyrrolidone, hydroxypropylmethylcellulose and half the specified amount of magnesium stearate. Blanks, 13 mm in diameter, are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape.

| | |
|---|---|
| Weight of core: | 230 mg |
| die: | 9 mm, convex |

The tablet cores thus produced are coated with a film consisting essentially of hydroxypropylmethylcellulose. The finished film-coated tablets are polished with beeswax.

Weight of coated tablet:245 mg.

### EXAMPLE 3

Tablets Containing 100 mg of Active Substance

Composition:

One tablet contains:

| | |
|---|---|
| active substance | 100.0 mg |
| lactose | 80.0 mg |
| corn starch | 34.0 mg |
| polyvinylpyrrolidone | 4.0 mg |
| magnesium stearate | 2.0 mg |
| | 220.0 mg |

Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.

| | |
|---|---|
| Weight of tablet: | 220 mg |
| Diameter: | 10 mm, biplanar, facetted on both sides |
| | and notched on one side. |

### EXAMPLE 4

Tablets Containing 150 mg of Active Substance

Composition:

One tablet contains:

| | |
|---|---|
| active substance | 50.0 mg |
| powdered lactose | 89.0 mg |
| corn starch | 40.0 mg |
| colloidal silica | 10.0 mg |
| polyvinylpyrrolidone | 10.0 mg |
| magnesium stearate | 1.0 mg |
| | 300.0 mg |

Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and are mixed with the specified amount of magnesium stearate. Tablets are pressed from the mixture.

| | |
|---|---|
| Weight of tablet: | 300 mg |
| die: | 10 mm, flat |

### EXAMPLE 5

Hard gelatine capsules containing 150 mg of active substance

One capsule contains:

| | |
|---|---|
| active substance | 50.0 mg |
| corn starch (dried) approx. | 80.0 mg |
| lactose (powdered) approx. | 87.0 mg |
| magnesium stearate | 3.0 mg |
| approx. | 420.0 mg |

Preparation:

The active substance is mixed with the excipients, passed through a screen with a mesh size of 0.75 mm and mixed until homogeneous using a suitable apparatus. The finished mixture is packed into size 1 hard gelatine capsules.

Capsule filling: approx. 320 mg

Capsule shell: size 1 hard gelatine capsule.

### EXAMPLE 6

Suppositories Containing 150 mg of Active Substance

One suppository contains:

| | |
|---|---|
| active substance | 150.0 mg |
| polyethyleneglycol 1500 | 550.0 mg |
| polyethyleneglycol 6000 | 460.0 mg |
| polyoxyethylene sorbitan monostearate | 840.0 mg |
| | 2,000.0 mg |

Preparation:

After the suppository mass has been melted the active substance is homogeneously distributed therein and the melt is poured into chilled moulds.

### EXAMPLE 7

Suspension Containing 50 mg of Active Substance

100 ml of suspension contain:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. water ad | 100 | ml |

Preparation:

The distilled water is heated to 70° C. The methyl and propyl p-hydroxybenzoates together with the glycerol and the sodium salt of carboxymethylcellulose are dissolved therein with stirring. The solution is cooled to ambient temperature and the active substance is added and homogeneously dispersed therein by stirring. After the sugar, the sorbitol solution and the flavouring have been added and dissolved, the suspension is evacuated with stirring to eliminate air.

5 ml of suspension contain 50 mg of active substance.

### EXAMPLE 8

Ampoules Containing 10 mg Active Substance

Composition:

Composition:

| | |
|---|---|
| active substance | 10.0 mg |
| 0.01 N hydrochloric acid q.s. | |
| double-distilled water ad | 2.0 ml |

Preparation:

The active substance is dissolved in the necessary amount of 0.01 N HCl, made isotonic with common salt, filtered sterilely and transferred into 2 ml ampoules.

### EXAMPLE 9

Ampoules Containing 50 mg of Active Substance

Composition:

Composition:

| | |
|---|---|
| active substance | 50.0 mg |
| 0.01 N hydrochloric acid q.s. | |
| double-distilled water ad | 10.0 ml |

Preparation:

The active substance is dissolved in the necessary amount of 0.01 N HCl, made isotonic with common salt, filtered sterilely and transferred into 10 ml ampoules.

### EXAMPLE 10

Capsules for Powder Inhalation Containing 5 mg of Active Substance

One capsule contains:

One capsule contains:

| | |
|---|---|
| active substance | 5.0 mg |
| lactose for inhalation | 15.0 mg |
| | 20.0 mg |

Preparation:

The active substance is mixed with lactose for inhalation. The mixture is packed into capsules in a capsule-making machine (weight of the empty capsule approx. 50 mg).

| weight of capsule: | 70.0 mg |
|---|---|
| size of capsule = | 3 |

### EXAMPLE 11

Solution for Inhalation for Hand-held Nebulisers Containing 2.5 mg Active Substance

One spray contains:

| | |
|---|---|
| active substance | 2.500 mg |
| benzalkonium chloride | 0.001 mg |
| 1N hydrochloric acid q.s. | |
| ethanol/water (50/50) ad | 15.000 mg |

Preparation:

The active substance and benzalkonium chloride are dissolved in ethanol/water (50/50). The pH of the solution is adjusted with IN hydrochloric acid. The resulting solution is filtered and transferred into suitable containers for use in hand-held nebulisers (cartridges). Contents of the container: 4.5 g

What is claimed is:

1. A compound of the formula 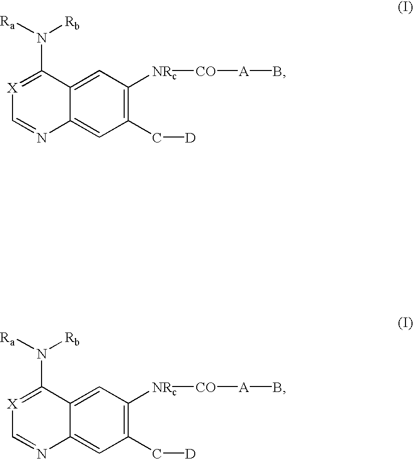
Rₐ denotes a hydrogen atom or a methyl group,
R_{b} denotes a phenyl, benzyl or 1-phenylethyl group, wherein the phenyl core is substituted in each case by the groups R₁ to R₃, whilst
R₁ and R₂, which may be identical or different, each denote a hydrogen, fluorine, chlorine, bromine or iodine atom,
a methyl, ethyl, hydroxy, methoxy, ethoxy, amino, cyano, vinyl or ethynyl group,
an aryl, aryloxy, arylmethyl or arylmethoxy group,
a methyl or methoxy group substituted by 1 to 3 fluorine atoms or
R₁ together with R₂, if they are bound to adjacent carbon atoms, denote a —CH═CH—CH═CH—, —CH═CH—NH— or —CH═N—NH— group and
R₃ denotes a hydrogen, fluorine, chlorine or bromine atom,
R_{c} denotes a hydrogen atom or a methyl group,
X denotes a methyne group substituted by a cyano group or a nitrogen atom,
A denotes a 1,1- or 1,2-vinylene group, each of which may be substituted by one or two methyl groups or by a trifluoromethyl group,
an ethynylene group, or
a 1,3-butadien-1,4-ylene group optionally substituted by a methyl or trifluoromethyl group,
B denotes a hydrogen atom or a C₁₋₄-alkyl group, a methyl group substituted by 1 to 3 fluorine atoms, an ethyl group substituted by 1 to 5 fluorine atoms, a C₁₋₄-alkylcarbonyl, carboxy, C₁₋₄-alkoxycarbonyl, aminocarbonyl, C₁₋₄-alkylaminocarbonyl, di-(C₁₋₄-alkyl)-aminocarbonyl, pyrrolidinocarbonyl, piperidinocarbonyl, morpholinocarbonyl or a 4-(C₁₋₄-alkyl)-piperazinocarbonyl group, or
a C₁₋₄-alkyl group substituted by the group R₄, whilst R₄ denotes a C₁₋₄-alkoxy group,
an amino group substituted by two C₁₋₄-alkyl groups, wherein the alkyl groups may be identical or different and each alkyl moiety may be substituted from position 2 by a C₁₋₄-alkoxy- or di-(C₁₋₄-alkyl)-amino group or by a 4- to 7-membered alkyleneimino group, whilst in the above-mentioned 6- to 7-membered alkyleneimino groups in each case a methylene group may be replaced in the 4-position by an oxygen or sulphur atom, by a sulphinyl, sulphonyl or N—(C₁₋₄-alkyl)-imino group,
a 4- to 7-membered alkyleneimino group optionally substituted by 1 to 4 methyl groups, a 6- to 7-membered alkyleneimino group optionally substituted by 1 or 2 methyl groups, wherein in each case a methylene group in the 4-position is replaced by an oxygen or sulphur atom, by a sulphinyl, sulphonyl or N—(C₁₋₂-alkyl)-imino group, or an imidazolyl group optionally substituted by 1 to 3 methyl groups,
C denotes a C₁₋₆-alkylene group, a —O—C₁₋₆-alkylene group, whilst the alkylene moiety is linked to the group D, or an oxygen atom, which may not be linked to a nitrogen atom of the group D, and D denotes a pyrrolidino group in which the two hydrogen atoms are replaced in the 2-position by a group E, wherein
E denotes a —CH₂—O—CO—CH₂—, —CH₂CH₂—O—CO—, —CH₂—O—CO—CH₂CH₂—, —CH₂CH₂—O—CO—CH₂— or —CH₂CH₂CH₂—O—CO— bridge optionally substituted by one or two C₁₋₂-alkyl groups,
a pyrrolidino group in which the two hydrogen atoms are replaced in the 3-position by a group F, wherein
F denotes a —O—CO—CH₂CH₂—, —CH₂—O—CO—CH₂—, —CH₂CH₂—O—CO—, —O—CO—CH₂CH₂CH₂—, —CH₂—O—CO—CH₂CH₂—, —CH₂CH₂—O—CO—CH₂—, —CH₂CH₂CH₂—O—CO—, —O—CO—CH₂—NR₅—CH₂—, —CH₂—O—CO—CH₂—NR₅—, —O—CO—CH₂—O—CH₂— or —CH₂—O—CO—CH₂—O—bridge optionally substituted by one or two C₁₋₂-alkyl groups, whilst
R₅ denotes a hydrogen atom or a C₁₋₄-alkyl group,
a piperidino or hexahydroazepino group, wherein the two hydrogen atoms are replaced in the 2-position by a group E, where E is as hereinbefore defined,
a piperidino or hexahydroazepino group, wherein in each case the two hydrogen atoms in the 3-position or in the 4-position are replaced by a group F, where F is as hereinbefore defined,
a piperazino- or 4-(C₁₋₄-alkyl)-piperazino group, wherein the two hydrogen atoms in the 2-position or in the 3-position of the piperazino ring are replaced by a group E, where E is as hereinbefore defined,
a pyrrolidino or piperidino group, wherein two vicinal hydrogen atoms are replaced by a —O—CO—CH₂——CH₂—O—CO—, —O—CO—CH₂CH₂—, —CH₂—O—CO—CH₂—, —CH₂CH₂—O—CO—, —O—CO—CH₂—NR₅— or —O—CO—CH₂—O— bridge optionally substituted by one or two C₁₋₂-alkyl groups, whilst R₅ is as hereinbefore defined and the heteroatoms of the above-mentioned bridges are not bound to the 2- or 5-position of the pyrrolidino ring and are not bound to the 2- or 6-position of the piperidino ring,
a piperazino or 4-(C₁₋₄-alkyl)-piperazino group, wherein a hydrogen atom in the 2-position together with a hydrogen atom in the 3-position of the piperazino ring are replaced by a —CH₂—O—CO—CH₂— or —CH₂CH₂—O—CO— bridge optionally substituted by one or two C₁₋₂-alkyl groups,
a piperazino group in which a hydrogen atom in the 3-position together with the hydrogen atom in the 4-position are replaced by a —CO—O—CH₂CH₂— or —CH₂—O—CO—CH₂— bridge optionally substituted by one or two C₁₋₂-alkyl groups, whilst in each case the left-hand end of the above-mentioned bridges is bound to the 3-position of the piperazino ring,
a pyrrolidino, piperidino or hexahydroazepino group substituted by the group R₆, wherein
R₆ denotes a 2-oxo-tetrahydrofuranyl, 2-oxo-tetrahydropyranyl, 2-oxo-1,4-dioxanyl or 2-oxo-4-(C₁₋₄-alkyl)-morpholinyl group optionally substituted by one or two C₁₋₂-alkyl groups,
a pyrrolidino group substituted in the 3-position by a 2-oxo-morpholino group, whilst the 2-oxo-morpholino group may be substituted by one or two C₁₋₂-alkyl groups,
a piperidino or hexahydroazepino group substituted in the 3- or 4-position by a 2-oxo-morpholino group, whilst the 2-oxo-morpholino group may be substituted by one or two C₁₋₂-alkyl groups,
a 4-(C₁₋₄-alkyl)-piperazino or 4-(C₁₋₄-alkyl)-homopiperazino group substituted at a ring nitrogen atom by R₆, wherein R₆ is as hereinbefore defined,
a piperazino or homopiperazino group substituted in the 4-position by the group R₇, wherein
R₇ denotes a 2-oxo-tetrahydrofuran-3-yl, 2-oxo-tetrahydrofuran-4-yl, 2-oxo-tetrahydropyran-3-yl, 2-oxo-tetrahydropyran-4-yl or 2-oxo-tetrahydropyran-5-yl group optionally substituted by one or two C₁₋₂-alkyl groups,
a pyrrolidino group substituted in the 3-position by a (R₅NR₇)—, R₇—, R₇S—, R₇SO— or R₇SO₂— group, whilst R₅ and R₇ are as hereinbefore defined,
a piperidino or hexahydroazepino group substituted in the 3- or 4-position by a (R₅NR₇)—, R₇O—, R₇S—, R₇SO— or R₇SO₂— group, wherein R₅ and R₇ are as hereinbefore defined,
a pyrrolidino, piperidino or hexahydroazepino group substituted by a R₆—C₁₋₄-alkyl-, (R₅NR₇)-C₁₋₄-alkyl-, R₇O—C₁₋₄-alkyl-, R₇S—C₁₋₄-alkyl-, R₇SO—C₁₋₄-alkyl-, R₇SO₂—C₁₋₄-alkyl- or (R₅NR₇)—CO— group, wherein R₅ to R₇ are as hereinbefore defined,
a pyrrolidino group substituted in the 3-position by a R₆—CO—NR₄, R₆-C₁₋₄-alkylene-CONR₄, (R₅NR₇)—C₁₋₄-alkylene-CONR₅, R₇O—C₁₋₄-alkylene-CONR₅, R₇S—C₁₋₄-alkylene-CONR₅, R₇SO—C₁₋₄-alkylene-CONR₅, R₇SO₂—C₁₋₄-alkylene-CONR₅, 2-oxo-morpholino-C₁₋₄-alkylene-CONR₅, R₆—C₁₋₄-alkylene-Y or C₂₋₄-alkyl-Y group, whilst the C₂₋₄-alkyl moiety of the C₂₋₄-alkyl-Y group is substituted in each case from position 2 by a (R₅NR₇)—, R₇O—, R₇S—, R₇SO— or R₇SO₂— group and the 2-oxo-morpholino moiety may be substituted by one or two C₁₋₂-alkyl groups, wherein
R₅ to R₇ are as hereinbefore defined and
Y denotes an oxygen or sulphur atom, an imino, N—(C₁₋₄-alkyl)-imino, sulphinyl or sulphonyl group,
a piperidino- or hexahydroazepino group substituted in the 3- or 4-position by a R₆—CO—NR₅, R₆—C₁₋₄-alkylene-CONR₅, (R₅NR₇)-C₁₋₄-alkylene-CONR₅, R₇O—C₁₋₄-alkylene-CONR₅, R₇S—C₁₋₄-alkylene-CONR₅, R₇SO—C₁₋₄-alkylene-CONR₅, R₇SO₂-C₁₋₄-alkylene-CONR₅, 2-oxo-morpholino-C 1-4-alkylene-CONR₅, R₆—C₁₋₄-alkylene-Y or C₂₋₄-alkyl-Y group, wherein Y is as hereinbefore defined, the 2-oxo-morpholino moiety may be substituted by one or two C₁₋₂-alkyl groups and the C₂₋₄-alkyl moiety of the C₂₋₄-alkyl-Y group is substituted in each case from position 2 by a (R₅NR₇)-, R₇O—, R₇S—, R₇SO— or R₇SO₂— group, whilst R₅ to R₇ are as hereinbefore defined,
a 4-(C₁₋₄-alkyl)-piperazino or 4-(C₁₋₄-alkyl)-homopiperazino group substituted at a ring nitrogen atom by a R₆-C₁₋₄-alkyl-, (R₅NR₇)-C₁₋₄-alkyl-, R₇O—C₁₋₄-alkyl-, R₇S—C₁₋₄-alkyl-, R₇SO—C₁₋₄-alkyl-, R₇SO₂-C₁₋₄-alkyl- or R₅NR₇—CO— group, wherein R₅ to R₇ are as hereinbefore defined,
a piperazino or homopiperazino group substituted in the 4-position by a R₆-C₁₋₄-alkyl-, R₆—CO—, R₆—C₁₋₄-alkylene-CO—, (R₅NR₇)—C₁₋₄-alkylene-CO—, R₇O—C₁₋₄-alkylene-CO—, R₇S—C₁₋₄-alkylene-CO—, R₇SO—C₁₋₄-alkylene-CO— or R₇SO₂—C₁₋₄-alkylene-CO— group, wherein R₅ to R₇ are as hereinbefore defined,
a piperazino or homopiperazino group substituted in the 4-position by a C₂₋₄-alkyl group, wherein the C₂₋₄-alkyl group is substituted in each case from position 2 by an (R₅NR₇)—, R₇O—, R₇S—, R₇SO— or R₇SO₂— group, whilst R₅ and R₇ are as hereinbefore defined,
a pyrrolidino, piperidino- or hexahydroazepino group substituted by a 2-oxo-morpholino-C₁₋₄-alkyl group, wherein the 2-oxo-morpholino moiety may be substituted by one or two C₁₋₂-alkyl groups,
a pyrrolidino group, substituted in the 3-position by a C₂₋₄-alkyl-Y group, wherein Y is as hereinbefore defined and the C₂₋₄-alkyl moiety of the C₂₋₄-alkyl-Y group is substituted in each case from position 2 by a 2-oxo-morpholino group optionally substituted by one or two C₁₋₂-alkyl groups,
a piperidino or hexahydroazepino group substituted in the 3- or 4-position by a C₂₋₄-alkyl-Y group, wherein Y is as hereinbefore defined and the C₂₋₄-alkyl moiety of the C₂₋₄-alkyl-Y group is substituted in each case from position 2 by a 2-oxo-morpholino group optionally substituted by one or two C₁₋₂-alkyl groups,
a 4-(C₁₋₄-alkyl)-piperazino- or 4-(C₁₋₄-alkyl)-homopiperazino group substituted at a ring nitrogen atom by a 2-oxo-morpholino-C₁₋₄-alkyl group, wherein the 2-oxo-morpholino moiety may be substituted by one or two C₁₋₂-alkyl groups,
a piperazino or homopiperazino group substituted in the 4-position by a 2-oxo-morpholino-C₁₋₄-alkylene-CO group, wherein the 2-oxo-morpholino moiety may be substituted by one or two C₁₋₂-alkyl groups,
a piperazino or homopiperazino group substituted in the 4-position by a C₂₋₄-alkyl group, wherein the C₂₋₄-alkyl moiety is substituted in each case from position 2 by a 2-oxo-morpholino group optionally substituted by one or two C₁₋₂-alkyl groups,
a pyrrolidinyl or piperidinyl group substituted in the 1-position by the group R₇, by a R₆—C₁₋₄-alkyl-, R₆—CO—, R₆—C₁₋₄-alkylene-CO—, (R₅NR₇)—C₁₋₄-alkylene-CO—, R₇O—C₁₋₄-alkylene-CO—, R₇S—C₁₋₄-alkylene-CO—, R₇SO—C₁₋₄-alkylene-CO—, R₇SO₂-C₁₋₄-alkylene-CO— or 2-oxo-morpholino-C₁₋₄-alkylene-CO— group, wherein R₅ to R₇ are as hereinbefore defined and the 2-oxo-morpholino moiety may be substituted by one or two C₁₋₂-alkyl groups,
a pyrrolidinyl or piperidinyl group substituted in the 1-position by a C₂₋₄-alkyl group, wherein the C₂₋₄-alkyl moiety is substituted in each case from position 2 by a (R₅NR₇)—, R₇O—, R₇S—, R₇SO—, R₇SO₂— or 2-oxo-morpholino group, whilst R₅ and R₇ are as hereinbefore defined and the 2-oxo-morpholino moiety may be substituted by one or two C₁₋₂-alkyl groups,
a pyrrolidin-3-yl-NR₅, piperidin-3-yl-NR₅ or piperidin-4-yl-NR₅ group substituted at the ring nitrogen atom in each case by the group R₇, by a R₆—C₁₋₄-alkyl-, R₆—CO—, R₆—C₁₋₄-alkylene-CO—, (R₅NR₇)—C₁₋₄-alkylene-CO—, R₇O—C₁₋₄-alkylene-CO—, R₇S—C₁₋₄-alkylene-CO—, R₇SO—C₁₋₄-alkylene-CO—, R₇SO₂-C₁₋₄-alkylene-CO— or 2-oxo-morpholino-C₁₋₄-alkylene-CO— group, wherein R₅ to R₇ are as hereinbefore defined and the 2-oxo-morpholino moiety may be substituted by one or two C₁₋₂-alkyl groups,
a pyrrolidin-3-yl-NR₅, piperidin-3-yl-NR₅ or piperidin-4-yl-NR₅ group substituted in each case at the ring nitrogen atom by a C₂₋₄-alkyl group, wherein the C₂₋₄-alkyl moiety is substituted in each case from position 2 by a (R₅NR₇)—, R₇O—, R₇S—, R₇SO—, R₇SO₂— or 2-oxo-morpholino group, whilst R₅ and R₇ are as hereinbefore defined and the 2-oxo-morpholino moiety may be substituted by one or two C₁₋₂-alkyl groups,
a R₆-C₁₋₄-alkylene-NR₅ group in which R₅ and R₆ are as hereinbefore defined, or
a C₂₋₄-alkyl-NR₄ group, wherein the C₂₋₄-alkyl moiety is substituted in each case from position 2 by a (R₅NR₇)—, R₇O—, R₇S—, R₇SO—, R₇SO₂— or 2-oxo-morpholino group, whilst R₅ and R₇ are as hereinbefore defined and the 2-oxo-morpholino moiety may be substituted by one or two C₁₋₂-alkyl groups,
a 2-oxo-morpholin-4-yl group substituted by the group R₈ or by the group R₈ and a C₁₋₄-alkyl group, whilst
R₈ denotes a C₃₋₄-alkyl, hydroxy-C₁₋₄-alkyl, C₁₋₄-alkoxy-C₁₋₄-alkyl, di-(C₁₋₄-alkyl)-amino-C₁₋₄-alkyl, pyrrolidino-C₁₋₄-alkyl, piperidino-C₁₋₄-alkyl, morpholino-C₁₋₄-alkyl, 4-(C₁₋₄-alkyl)-piperazino-C₁₋₄-alkyl, C₁₋₄-alkylsulphanyl-C₁₋₄-alkyl, C₁₋₄-alkylsulphinyl-C₁₋₄-alkyl, C₁₋₄-alkylsulphonyl-C₁₋₄-alkyl, cyan-C₁₋₄-alkyl, C₁₋₄-alkoxycarbonyl-C₁₋₄-alkyl, aminocarbonyl-C₁₋₄-alkyl, C₁₋₄-alkyl-aminocarbonyl-C₁₋₄-alkyl, di-(C₁₋₄-alkyl)-aminocarbonyl-C₁₋₄-alkyl, pyrrolidinocarbonyl-C₁₋₄-alkyl, piperidinocarbonyl-C₁₋₄-alkyl, morpholinocarbonyl-C₁₋₄-alkyl or a 4-(C₁₋₄-alkyl)-piperazinocarbonyl-C₁₋₄-alkyl group,
a 2-oxo-morpholin-4-yl group substituted by two groups R₈, whilst R₈ is as hereinbefore defined and the two groups R₈ may be identical or different,
a 2-oxo-morpholin-4-yl group in which the two hydrogen atoms of a methylene group are replaced by a —(CH₂)ₘ—, —CH₂—Y—CH₂—, —CH₂—Y—CH₂—CH₂—, —CH₂CH₂—Y—CH₂CH₂— or —CH₂CH₂—Y—CH₂CH₂CH₂— bridge optionally substituted by one or two C₁₋₂-alkyl groups, whilst
m denotes the number 2, 3, 4, 5 or 6 and
Y denotes an oxygen or sulphur atom, a sulphinyl, sulphonyl or C₁₋₄-alkylimino group,
a 2-oxo-morpholin-4-yl group in which a hydrogen atom in the 5-position together with a hydrogen atom in the 6-position is replaced by a —(CH₂)ₙ—, —CH₂—Y—CH₂—, —CH₂—Y—CH₂CH₂— or —CH₂—CH₂—Y—CH₂— bridge, whilst
Y is as hereinbefore defined and
n denotes the number 2, 3 or 4,
whilst, unless otherwise stated, the aryl moieties mentioned in the definitions of the above-mentioned groups denote a phenyl group which may be mono- or disubstituted by R₉, whilst the substituents may be identical or different and
R₉ denotes a fluorine, chlorine, bromine or iodine atom, a C₁₋₂-alkyl, trifluoromethyl or C₁₋₂-alkoxy group, or
two groups R₉, if they are bound to adjacent carbon atoms, together denote a C₃₋₄-alkylene, methylenedioxy or 1,3-butadien-1,4-ylene group, or a tautomer or salt thereof.

2. A compound of the formula I according to claim 1, wherein
Rₐ denotes a hydrogen atom,
R_{b} denotes a 1-phenylethyl, 3-methylphenyl, 3-chlorophenyl, 3-bromophenyl or 3-chloro-4-fluorophenyl group,
R_{c} denotes a hydrogen atom,
X denotes a nitrogen atom,
A denotes a 1,2-vinylene or ethynylene group,
B denotes a hydrogen atom,
C denotes an —O—CH₂CH₂—, —O—CH₂CH₂CH₂— or —O—CH₂CH₂CH₂CH₂— group, whilst the alkylene moiety in each case is linked to the group D, and
D denotes a piperidino group in which the two hydrogen atoms in the 4-position are replaced by a —CH₂—O—CO—CH₂, —CH₂CH₂—O—CO—, —CH₂CH₂—O—CO—CH₂—, —O—CO—CH₂—NCH₃—CH₂— or —O—CO—CH₂—O—CH₂— bridge,
a piperazino group in which a hydrogen atom in the 3-position together with the hydrogen atom in the 4-position are replaced by a —CO—O—CH₂—CH₂— or —CH₂—O—CO—CH₂— bridge, whilst in each case the left-hand ends of the above-mentioned bridges are bound to the 3-position of the piperazino ring,
a piperidino group which is substituted in the 4-position by a 2-oxo-morpholino or 2-oxo-morpholinomethyl group, whilst the 2-oxo-morpholino moiety may be substituted in each case by one or two methyl groups,
a piperazino group which is substituted in the 4-position by a 2-oxo-tetrahydrofuran-3-yl- or 2-oxo-tetrahydrofuran-4-yl group,
a piperidino group which is substituted in the 4-position by a R₆S group, whilst
R₆ denotes a 2-oxo-tetrahydrofuran-3-yl or 2-oxo-tetrahydrofuran-4-yl group,
a piperazino group which is substituted in the 4-position by a 2-oxo-tetrahydrofuranylmethyl or 2-oxo-tetrahydrofuranyl-carbonyl group,
a piperazino group which is substituted in the 4-position by a [2-(2-oxo-tetrahydrofuran-3-ylsulphenyl)ethyl] group,
a piperidin-4-yl group which is substituted in the 1-position by a 2-oxo-tetrahydrofuran-3-yl or 2-oxo-tetrahydrofuran-4-yl group,
a 2-oxo-morpholin-4-yl group which is substituted by a methoxymethyl or methoxyethyl group,
a 2-oxo-morpholin-4-yl group in which the two hydrogen atoms of a methylene group are replaced by a —CH₂CH₂CH₂CH₂—, —CH₂CH₂CH₂CH₂CH₂—, —CH₂—O—CH₂CH₂— or —CH₂CH₂—O—CH₂CH₂— bridge, or a tautomer or salt thereof.

3. A compound of the formula I according to claim 1, wherein
Rₐ denotes a hydrogen atom,
R_{b} denotes a 1-phenylethyl or 3-chloro-4-fluorophenyl group,
R_{c} denotes a hydrogen atom,
X denotes a nitrogen atom,
A denotes a 1,2-vinylene group,
B denotes a hydrogen atom,
C denotes an —O—CH₂CH₂—, —O—CH₂CH₂CH₂— or —O—CH₂CH₂CH₂CH₂— group, whilst the alkylene moiety in each case is linked to the group D, and
D denotes a piperazino group which is substituted in the 4-position by a 2-oxo-tetrahydrofuran-4-yl or 2-oxo-tetrahydrofuran-5-ylcarbonyl group,
or a tautomer or salt thereof.

4. A compound selected from the group consisting of:
(1) 4-[(3-chloro-4-fluorophenyl)amino]-7-{3-[4-(2-oxo-tetrahydrofuran-4-yl)-piperazin-1-yl]-propyloxy}-6-[(vinylcarbonyl)amino]-quinazoline,
(2) 4-[(3-chloro-4-fluorophenyl)amino]-7-(2-{4-[(S)-(2-oxo-tetrahydrofuran-5-yl)carbonyl]-piperazin-1-yl}-ethoxy)-6-[(vinylcarbonyl)amino]-quinazoline,
(3) 4-[(R)-(1-phenylethyl)amino]-7-{2-[4-(2-oxo-tetrahydrofuran-4-yl)-piperazin-1-yl]-ethoxy}-6-[(vinylcarbonyl)amino]-quinazoline and
(4) 4-[(3-chloro-4-fluorophenyl)amino]-7-{2-[4-(2-oxo-tetrahydrofuran-4-yl)-piperazin-1-yl]-ethoxy}-6-[(vinylcarbonyl)amino]-quinazoline,
or a tautomer or salt thereof.

5. A physiologically acceptable salt of a compound according claim 1, 2, 3, or 4, formed with an inorganic or organic acid or base.

6. A pharmaceutical composition containing a compound according claim 1, 2, 3, or 4 or a pharmaeceutically acceptable salt thereof, and a pharmaceutically acceptable carrier or diluent.

7. A method of treating a benign or malignant tumour, a disease of the respiratory tract or lungs, polyps, a disease of the gastro-intestinal tract, bile duct or gall bladder, a disease of the kidneys or of the skin, which comprises administering a therapeutically effective amount of a compound according claim 1, 2, 3, or 4 or a pharmaceutically acceptable salt thereof.
